(19) United States
(12) Patent Application Publication
(10) Pub. No.: US 2026/0354386 A1
(43) Pub. Date: Nov. 12, 2026

(54) INSECTICIDAL FUSION POLYPEPTIDES AND METHODS FOR THEIR USE

(71) Applicant: IMPETUS AGRICULTURE, INC., St. Louis, MO (US)

(72) Inventors: Heba Mohamed Yassen ABDELGAFFAR, Knoxville, TN (US); Juan Luis JURAT-FUENTES, Knoxville, TN (US); Alexander Eugene HROMOCKYJ, Chesterfield, MO (US)

(21) Appl. No.: 19/486,269

(22) PCT Filed: May 31, 2024
(86) PCT No.: PCT/US2024/031903

§ 371 Date: Nov. 20, 2025

Related U.S. Application Data
(60) Provisional application No. 63/505,286, filed on May 31, 2023.
(60) Provisional application No. 63/505,949, filed on Jun. 2, 2023.

(51) Int. Cl. A01N 63/50; A01P 7/04; C07K 14/435; C07K 16/18; C12N 15/82

(52) U.S. Cl. CPC A01N 63/50; A01P 7/04; C07K 14/43563; C07K 16/18; C12N 15/8286; C07K 2317/569; C07K 2319/55

(57) ABSTRACT

Insecticidal fusion polypeptides comprising Cry1 protoxin and nanobody fusion proteins and their targets are disclosed. Also disclosed are the nucleic acid molecules that encode them, cells comprising said constructs, as well as compositions and methods for controlling plant pests.

### CROSS REFERENCE TO RELATED APPLICATIONS

This International patent application claims the benefit of U.S. Provisional patent application Ser. No. 63/505,949, filed Jun. 2, 2023; and the benefit of U.S. Provisional patent application Ser. No. 63/505,286, filed May 31, 2023.

### REFERENCE TO SEQUENCE LISTING SUBMITTED ELECTRONICALLY

The instant application contains a Sequence Listing named “P14387WO00.xml” which has been submitted electronically in XML format, is 225,874 bytes in size, was created on May 31, 2024, and is herein incorporated by reference in its entirety. Also incorporated herein by reference in its entirety is the XML copy of the sequence listing submitted in U.S. Provisional patent application Ser. No. 63/505,949, created on Jun. 2, 2023, named “P14387US01.xml” and which is 215,558 bytes in size.

### TECHNICAL FIELD

The present disclosure relates generally to the field of insecticidal proteins and their targets, the nucleic acid molecules that encode them, as well as compositions and methods for controlling plant pests.

### BACKGROUND

There is a need in the art to develop new and effective pest control agents that provide an economic benefit to farmers and that are environmentally acceptable. Particularly needed are control agents that can target to a wider spectrum of insect pests and that efficiently control insect strains that are or could become resistant to existing insect control agents.

Certain species of microorganisms of the genus Bacillus are known to possess pesticidal activity against a range of insect pests. Bacillus thuringiensis (Bt) is a gram-positive spore forming soil bacterium characterized by its ability to produce crystalline inclusions that are specifically toxic to certain orders and species of plant pests, including insects, but are harmless to plants and other non-target organisms. For this reason, compositions with Bacillus thuringiensis strains, or their insecticidal proteins can be used as environmentally acceptable insecticides to control agricultural insect pests or insect vectors of a variety of human or animal diseases. Crystal (Cry) proteins from Bacillus thuringiensis have potent insecticidal activity against predominantly Lepidoptera, Diptera, Coleoptera, Hemiptera and Nematode pests. Based on this property crop plants have been genetically engineered to produce insecticidal proteins from Bacillus thuringiensis to thereby exhibit enhanced insect resistance. Sprays and surface applications of microbial insecticides provide an environmentally friendly alternative to synthetic chemical pesticides and can be produced in a cost-effective manner.

A serious threat to the continued efficacy of current insecticidal proteins, such as Cry proteins, whether expressed in transgenic plants or applied over the top on crops or on insect pests, is the evolution of resistance in target pests (Tabashnik et al. 2013, Nat Biotechnol 31, 510-521; Tabashnik and Carriere, 2017 Nat Biotechnol. 35(10):926-935). At least seven different insect species have developed field-evolved resistance to several Bt toxins, such as Cry toxins, that has practical consequences for pest control (practical resistance) in transgenic crops. The most common resistance mechanism is the reduction in toxin binding to midgut cells, that in different insect species include mutations in Cry toxin receptors such as cadherin, ABC transporters, and aminopeptidase (APN) (reviewed in Pardo-Lopez et al. 2013, FEMS Microbiol. Rev. 37, 3-22; Fabrick and Wu, 2023, Advances in Insect Physiology. 65, 123-183.).

To date, approaches to address Cry toxin insect resistance have focused on identification of new toxins or targeted modification of existing toxins. More than 1,100 toxin genes, encoding different entomopathogenic proteinaceous toxins, have been identified and characterized in the Bt strains isolated from different regions of the world (on the world wide web internet site “lifesci.sussex.ac.uk/Home/Neil_Crickmore/Bt/” and Crickmore at al. on the world wide web internet site “bpprc.org” as viewed on May 30, 2024). The toxins have very different activity spectra against various insect classes and nematodes. Therefore, identifying new toxins against specific targets is tedious, often non-targeted and requires large-scale screens with limited probability of success. Modifications of existing Cry toxins have mainly been limited to specific protein domains that are required for binding to the target sequence (domains II and III; DII and DIII, see FIG. 1), because other modifications can reduce the stability of the toxins, reduce their specificity, or interfere with the mechanisms of toxicity, such as pore formation (domain I, DI) in the insect gut.

### SUMMARY

Polypeptides comprising from N-terminus to C-terminus, (a) domain I (DI), domain II (DII), and domain III (DIII) of a Cry1 protein; (b) a protease cleavage site; (c) a linker peptide; (d) a nanobody; and (e) a C-terminal Cry1 protoxin domain, wherein said elements are operably linked are provided. Polynucleotides encoding the polypeptides are also provided. Cells comprising the polypeptides or polynucleotides are also provided. Polypeptides comprising from N-terminus to C-terminus, polypeptides comprising domain I (DI), domain II (DII), and domain III (DIII) of a Cry1 protein, a protease cleavage site, an N-terminal segment of a Cry1 protoxin domain, a linker peptide, a nanobody; and a C-terminal segment of a Cry1 protoxin domain, wherein the polypeptides are operably linked are also provided.

Plants comprising a polypeptide comprising from N-terminus to C-terminus, (a) domain I (DI), domain II (DII), and domain III (DIII) of a Cry1 protein; (b) a protease cleavage site; (c) a linker peptide; (d) a nanobody; and (e) a C-terminal Cry1 protoxin domain, wherein said elements are operably linked or other polypeptides disclosed herein are provided.

Methods of controlling Lepidopteran insects are also provided. In some embodiments, the method comprises the step of cultivating plants treated with a composition comprising a polypeptide comprising from N-terminus to C-terminus, (a) domain I (DI), domain II (DII), and domain III (DIII) of a Cry1 protein; (b) a protease cleavage site; (c) a linker peptide; (d) a nanobody; and (e) a C-terminal Cry1 protoxin domain, wherein said elements are operably linked, in the presence of Lepidopteran insects which feed on susceptible varieties of the plant.

Biological samples comprising a polypeptide comprising from N-terminus to C-terminus, (a) domain I (DI), domain II (DII), and domain III (DIII) of a Cry1 protein; (b) a protease cleavage site; (c) a linker peptide; (d) a nanobody; and (e) a C-terminal Cry1 protoxin domain, wherein said elements are operably linked or other polypeptides disclosed herein are also provided. Biological samples comprising the polynucleotide encoding the polypeptide are also provided.

Compositions comprising a polypeptide comprising from N-terminus to C-terminus, (a) domain I (DI), domain II (DII), and domain III (DIII) of a Cry1 protein; (b) a protease cleavage site; (c) a linker peptide; (d) a nanobody; and (e) a C-terminal Cry1 protoxin domain, wherein said elements are operably linked or other polypeptides disclosed herein, and at least one additional ingredient are provided. Methods of controlling Lepidopteran insects comprising applying the composition to plants susceptible to Lepidopteran insect feeding are also provided. Plants and plant parts which are coated or partially coated with the compositions are also provided.

Compositions comprising a cell comprising a polypeptide comprising from N-terminus to C-terminus, (a) domain I (DI), domain II (DII), and domain III (DIII) of a Cry1 protein; (b) a protease cleavage site; (c) a linker peptide; (d) a nanobody; and (e) a C-terminal Cry1 protoxin domain, wherein said elements are operably linked or other polypeptides disclosed herein, and at least one additional ingredient are provided. Methods of controlling Lepidopteran insects comprising applying the composition to plants susceptible to Lepidopteran insect feeding are also provided. Plants and plant parts which are coated or partially coated with the compositions are also provided.

Methods of producing Bacillus thuringiensis spores comprising a polypeptide comprising from N-terminus to C-terminus, (a) domain I (DI), domain II (DII), and domain III (DIII) of a Cry1 protein; (b) a protease cleavage site; (c) a linker peptide; (d) a nanobody; and (e) a C-terminal Cry1 protoxin domain or other polypeptides disclosed herein, wherein said elements are operably linked as a protoxin crystal are provided. In some embodiments, the method comprises the step of culturing Bacillus thuringiensis in media which induces spore formation and harvesting spores from the media.

### BRIEF DESCRIPTION OF THE DRAWINGS

FIG. 1 is a diagrammatic representation (A) the generic structure of a Bt Cry1 protein protoxin consisting of a N-terminal protoxin peptide, three domain (DI, DII, DIII) core toxin, a serine protease cleavage site, and the C-terminal protoxin protein moiety. (B) prototypical structure of active Cry protein protoxin::affinity proteins consisting of a linker and affinity protein inserted at a position in the protoxin located downstream of the C-terminus of DIII.

FIG. 2 shows sporulation and protoxin crystal formation. Phase contrast images of example cultures of Bt strain 4D22 transformants of: (A) wild-type toxin (SEQ ID NO: 2; normal crystals); (B) “active” fusion construct (SEQ ID NO: 53; larger crystals); and (C) Domain II (DII) fusion construct (SEQ ID NO: 51; no crystals).

### DETAILED DESCRIPTION

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “comprise,” “comprises,” “comprising,” “include,” “includes,” and “including” are to be construed as at least having the features to which they refer while not excluding any additional unspecified features.

As used herein, “heterologous” refers to a polynucleotide or peptide sequence located in, e.g., a genome or a vector, in a context other than that in which the sequence occurs in nature. For example, a promoter that is operably linked to a gene other than the gene that the promoter is operably linked to in nature is a heterologous promoter.

As used herein, the phrase “operably linked” refers to a juxtaposition wherein the components so described are in a relationship permitting them to function in their intended manner. For instance, a promoter is operably linked to a coding sequence if the promoter provides for transcription or expression of the coding sequence.

As used herein the term “percent identity” refers to percent (%) sequence identity with respect to a reference polynucleotide or polypeptide sequence following alignment by standard techniques. Alignment for purposes of determining percent nucleic acid or amino acid sequence identity can be achieved in various ways that are within the capabilities of one of skill in the art, for example, using publicly available computer software such as BLAST, BLAST-2, PSI-BLAST, or Megalign software. In some embodiments, the software is MUSCLE (Edgar, Nucleic Acids Res., 32(5): 1792-1797, 2004). Those skilled in the art can determine appropriate parameters for aligning sequences, including any algorithms needed to achieve maximal alignment over the full length of the sequences being compared. For example, in embodiments, percent sequence identity values are generated using the sequence comparison computer program BLAST (Altschul et al. (1990) J. Mol. Biol., 215:403-410). As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows: 100 multiplied by (the fraction X/Y), where X is the number of nucleotides or amino acids scored as identical matches by a sequence alignment program (e.g., BLAST) in that program's alignment of A and B, and where Y is the total number of nucleotides or amino acids in B.

As used herein, the term “plant” includes a whole plant and any descendant, cell, tissue, or part of a plant. The term “plant parts” include any part(s) of a plant, including, for example and without limitation: seed (including mature seed and immature seed); a plant cutting; a plant cell; a plant cell culture; or a plant organ (e.g., pollen, embryos, flowers, fruits, shoots, leaves, roots, stems, and explants). A plant tissue or plant organ can be a seed, protoplast, callus, or any other group of plant cells that is organized into a structural or functional unit. A plant cell or tissue culture can be capable of regenerating a plant having the physiological and morphological characteristics of the plant from which the cell or tissue was obtained, and of regenerating a plant having substantially the same genotype as the plant. Regenerable cells in a plant cell or tissue culture can be embryos, protoplasts, meristematic cells, callus, pollen, leaves, anthers, roots, root tips, flowers, or stalks. In contrast, some plant cells are not capable of being regenerated to produce plants and are referred to herein as “non-regenerable” plant cells.

As used herein, the phrase “plant cell” can refer either a plant cell having a plant cell wall or to a plant cell protoplast lacking a plant cell wall.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

Unless otherwise stated, nucleic acid sequences described herein are given, when read from left to right, in the 5′ to 3′ direction. Nucleic acid sequences can be provided as DNA or as RNA, as specified. Furthermore, because of known codon degeneracy, different nucleic acid sequences can encode the same polypeptide sequence, and such modified nucleic acid sequences (e.g., for the purposes of codon optimization for a given species) are within the scope of the present disclosure. Where a term is provided in the singular, it also contemplates aspects of the invention described by the plural of that term.

The present disclosure provides Cry toxin fusion polypeptides which can target different insect protein receptors while retaining their ability to form parasporal crystals. As a result, toxin activity against Cry toxin resistant populations is enhanced and the spectrum of Cry toxin activity is expanded to insect species that are naturally less susceptible to their mechanism of action.

The present disclosure provides, inter alia, insecticidal fusion polypeptides and related systems, methods, and compositions. In certain embodiments, high affinity molecules (nanobodies) have been inserted in Cry protoxin protein sequences to create Cry1 toxin::nanobody fusion polypeptide constructs. Accordingly, in some embodiments, the fusion polypeptide is a Cry1 toxin::nanobody fusion polypeptide construct.

In certain embodiments, the insecticidal fusion polypeptide is a polypeptide comprising, from N-terminus to C-terminus, domain I (DI), domain II (DII), and domain III (DIII) of a Cry protein, a protease cleavage site, a linker peptide, a nanobody, and a C-terminal Cry protoxin domain, wherein said elements are operably linked. In some embodiments, the Cry protein is a Cry1 protein. In some embodiments, the Cry protoxin domain is a Cry1 protoxin domain. Representative Cry1 A proteins which can be used in the polypeptides provided herein include the following (with accession numbers for their sequences from the world wide web internet site “ncbi.nlm.nih.gov/protein”): Cry1Aa (e.g., Cry1Aa1, Accession #M11250), Cry1Ab (e.g., Cry1Ab1, Accession #M13898), Cry1Ab-like (Accession #AF327924 or #AF327925 or #AF327926), Cry1Ac (e.g., Cry1Ac1, Accession #M11068), Cry1Ad (e.g., Cry1Ad1, Accession #M73250), Cry1Ae (e.g., Cry1Ae1, Accession #M65252), Cry1Af (e.g., Cry1Af1, Accession #U82003), Cry1Ag (e.g., Cry1Ag1, Accession #AF081248), Cry1Ah (e.g., Cry1Ah1, Accession #AF281866), Cry1Ai (e.g., Cry1Ai1, Accession #AY174873), Cry1A-Iike (Accession #AF327927), Cry1Ba (e.g., Cry1Ba1, Accession #X06711), Cry1Bb (e.g., Cry1Bb1, Accession #L32020), Cry1Bc1 (e.g., Cry1Bc1, Accession #Z46442), Cry1Bd (e.g., Cry1Bd1, Accession #U70726), Cry1Be (e.g., Cry1Be1, Accession #AF077326), Cry1Bf (e.g., Cry1Bf1, Accession #AX189649), Cry1Bg (e.g., Cry1Bg1, Accession #AY176063), Cry1Ca (e.g., Cry1Ca1, Accession #X07518), Cry1Cb (e.g., Cry1Cb1, Accession #M97880), Cry1Cb-Iike (Accession #AAX63901), Cry1 Da (e.g., Cry1Da1, Accession #X54160), Cry1Db (e.g., Cry1Db1, Accession #Z22511), Cry1Dc (e.g., Cry1Dc1, Accession #EF059913), Cry1Ea (e.g., Cry1Ea1, Accession #X53985), Cry1Eb (e.g., Cry1Eb1, Accession #M73253), Cry1Fa (e.g., Cry1Fa1, Accession #M63897), Cry1Fb (e.g., Cry1Fb1, Accession #Z22512), Cry1Ga (e.g., Cry1Ga1, Accession #Z22510), Cry1Gb (e.g., Cry1Gb1, Accession #U70725), Cry1Gc (Accession #AAQ52381), Cry1Ha (e.g., Cry1Ha1, Accession #Z22513), Cry1Hb (e.g., Cry1Hb1, Accession #U35780), Cry1H-Iike (Accession #AF182196), Cry1Ia (e.g., Cry1Ia1, Accession #X62821), Cry11b (e.g., Cry11b1, Accession #U07642), Cry11c (e.g., Cry11c1, Accession #AF056933), Cry11d (e.g., Cry11d1, Accession #AF047579), Cry11e (e.g., Cry11e1, Accession #AF211190), Cry11f (e.g., Cry11f1, Accession #AAQ52382), Cry11-Iike (Accession #190732), Cry1Ja (e.g., Cry1Ja1 (Accession #L32019), Cry1Jb (e.g., Cry1Jb1, Accession #U31527), Cry1Jc (e.g., Cry1Jc1 (Accession #I90730), Cry1Jd (e.g., Cry1Jd1 (Accession #AX189651), Cry1Ka (e.g., Cry1Ka1, Accession #U28801), Cry1La (e.g., Cry1La1, Accession #AAS60191), and Cry1-Iike (Accession #190729). In certain embodiments, the Cry1 protein or portion thereof (e.g., DI, DII, DIII, and/or protoxin domain) used in the insecticidal fusion polypeptide is the Cry1F protein or portion thereof set forth in SEQ ID NO: 3, 80, 81, 82, 83, 84, 85, 86, 87, 88, 89, or 90 in a chimera thereof, or in a peptide having at least 85%, 90%, 95%, 98%, or 99% sequence identity thereto. In certain embodiments, the Cry1 protoxin domain is a Cry1Ac, Cry1Ab, or Cry1Fa protoxin domain.

In certain embodiments, DI, DII, and DIII comprise the DI, DII, and DIII of a Cry protein. Structurally, Cry toxins comprise three distinct domains which are, from the N to C-terminus: a cluster of seven alpha-helices implicated in pore formation (referred to as “DI”), three anti-parallel beta sheets implicated in cell binding (referred to as “DII”), and a beta sandwich (referred to as “DIII”). The location and properties of these domains in Cry proteins have been described. (Li et al. (1991) Nature, 353:815-821; Morse et al. (2001) Structure, 9:409-417; U.S. Pat. No. 9,284,573, incorporated herein by reference in its entirety). In certain embodiments, DI, DII, and DIII comprise the DI, DII, and DIII of a Cry1 protein. In some embodiments, DI, DII, and DIII comprise the DI, DII, and DIII of a Cry1Aa, Cry1Ab, Cry1Ac, Cry1Fa, or Cry1Fb protein. In some embodiments, DI, DII, and DIII are all selected from the same Cry1 protein. In some embodiments, the Cry1 protein comprises a chimeric Cry1 protein wherein DI, DII, and DIII are selected from at least a first Cry1 protein and a second Cry1 protein. For example, DI and DII can be selected from Cry1Ac and DIII can be selected from Cry1F (e.g., Cry1Fa or Cry1Fb). In certain embodiments, the polypeptide is a Cry1Ac1Ac1F chimera which comprises the DI and DII of Cry1Ac or a polypeptide having at least 85%, 90%, 95%, 98%, or 99% sequence identity thereto and the DIII of a Cry1Fa protein set forth in SEQ ID NO: 3, 80, 81, 82, or 83, in a chimera thereof, or in a polypeptide having at least 85%, 90%, 95%, 98%, or 99% sequence identity thereto. In certain embodiments, the Cry1Ac1Ac1F chimera comprises the polypeptide of SEQ ID NO: 2, of SEQ ID NO: 89, a variant thereof comprising a polypeptide having at least 85%, 90%, 95%, 98%, or 99% sequence identity thereto, or a variant thereof disclosed in U.S. Pat. No. 6,521,442, which is incorporated herein by reference in its entirety.

In certain embodiments, the polypeptide comprises a protease cleavage site (e.g., a serine protease cleavage site). Such protease cleavage sites can include sites cleaved by trypsins, chymotrypsins, and elastases found in the gut of Lepidopteran insects (Lenz et al. (1991) Arch. Insect Biochem. Physiol. 16: 201-212; Hedegus et al. (2003) Arch. Insect Biochem. Physiol. 53: 30-47; Gatehouse et al. (1997) Insect Biochem. Mol. Biol. 27: 929-944; Peterson et al. (1995) Insect Biochem. Mol. Biol. 25: 765-774; Oppert (1999) Arch. Insect Biochem. Phys. 42: 1-12; and Carroll et al. (1997) J. Invertebrate Pathology 70: 41-49; U.S. Pat. No. 1,000,076, incorporated herein by reference in its entirety). In some embodiments, the protease cleavage site is a serine protease or a trypsin cleavage site. In certain embodiments, the protease, serine protease, or trypsin cleavage site is encoded by a polynucleotide located at the 3′ end of the DIII coding sequence or located 3′ to the 3′ end of the DIII coding sequence (e.g., one, two, three, four, or more codons are present between the 3′ end of the DIII coding sequence and the polynucleotide encoding the protease, serine protease, or trypsin cleavage site). In certain embodiments, the protease, serine protease, or trypsin cleavage site is located at the C-terminal end of the DIII coding sequence or located one, two, three, four, or more amino acids C-terminal to the DIII coding sequence.

In certain embodiments, the polypeptide comprises a linker peptide. In some embodiments, the linker peptide is a repeat of glycine residues. The linker peptide can be a repeat of at least 4, at least 5, at least 6, at least 7, at least 8, at least 9, at least 10, at least 11, or at least 12 glycine residues. In some embodiments, the linker peptide comprises a repeat of 8 glycine residues. In certain embodiments, the linker comprises or consists of eight (8) glycine residues (SEQ ID NO: 37). In other embodiments, the linker can comprise one or more (Gly)3-X and/or (Gly)4-X peptides, where X is serine, threonine, alanine, or asparagine. In certain embodiments, (Gly)3-Ser linkers (e.g., of three distinct lengths such as 9, 25, 35 amino acids) which contain glycine for flexibility and serine for solubility are used. Non-limiting examples of linkers comprising (Gly)4-X peptides include those comprising the polypeptide of SEQ ID NO: 35. In still other embodiments, the linker comprises proline and threonine residues (e.g., a peptide comprising the sequence PTPTn, (SEQ ID NO: 90; where n is 1, 2, 3, or more). Non-limiting examples of a proline threonine linker include SEQ ID NO: 39. In still other embodiments, the linker comprises an AEAAAK3 (SEQ ID NO: 31) linker or an ESGSV linker (SEQ ID NO: 33).

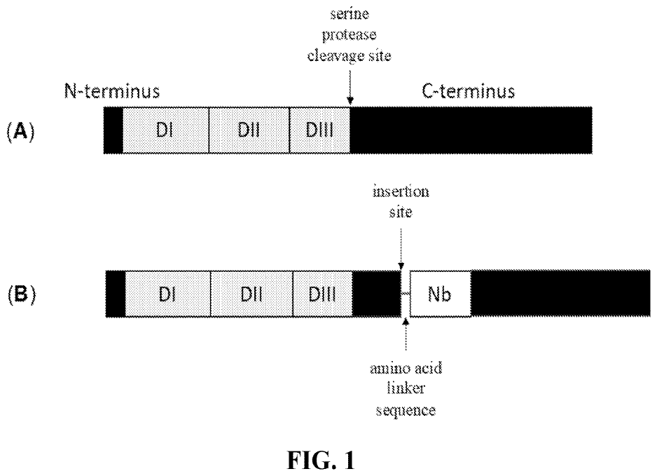

In certain embodiments, the polypeptide comprises a nanobody. In some embodiments, the nanobodies are isolated from a camelid (e.g., llama) immunized with peptide sequences of proteins expressed on the apical cell surface of an insect (e.g., a lepidopteran insect such as Spodoptera frugiperda, Fall armyworm; FAW) midgut epithelial cells. In certain embodiments, the nanobody was obtained by using an insect target protein not previously identified as a Cry1 toxin receptor as an immunogen or affinity probe to isolate the nanobody. In certain embodiments, the nanobody is obtained by using an insect (e.g., a lepidopteran insect such as FAW) nutrient amino acid transporter (NAAT) protein as an immunogen or affinity probe. In some embodiments, the nanobody which binds to a NAAT protein comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 5, 7, 9, 11, 13, 15, 17, 19, or 21. In certain embodiments, the nanobody is obtained by using a Cadherin (CAD) protein as an immunogen or affinity probe. In some embodiments, the nanobody which binds to a CAD protein comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 23, 25, 27, or 29. The polypeptide can comprise different combinations of linker peptides and nanobody combinations inserted at different locations within the Cry1 protoxin domain (e.g., as illustrated in FIG. 1). In certain embodiments, the linker peptide and nanobody is inserted C-terminal to an N-terminal segment of a Cry1 protoxin domain, where the N-terminal segment can comprise at least about 10, 20, 30, 40, 50, 60, 70, 80, 90, 100, 110, 120, 130, 140, or 150 amino acids of the N-terminal amino acids of the Cry1 protoxin domain. In certain embodiments, the N-terminal segment of the protoxin domain (e.g., comprising at least 80, 90, 100, 110, 120, 130, 140, or 150 amino acids of the N-terminal amino acids of the Cry1 protoxin domain) comprises an intact N-terminal protoxin domain amino acid sequence or an N-terminal protoxin domain amino sequence lacking 1, 2, 3, 4, 5 or more residues from the native protoxin domain N-terminal segment N- and/or C-terminus. In certain embodiments, the linker peptide and nanobody is inserted N-terminal to a C-terminal segment of a Cry1 protoxin domain, where the C-terminal segment can comprise at least about 400, 450, or 500 amino acids of the C-terminal segment of the Cry1 protoxin domain. In certain embodiments, the C-terminal segment of the protoxin domain (e.g., comprising at least 400, 450, or 500 amino acids of the C-terminal amino acids of the Cry1 protoxin domain) comprises an intact C-terminal protoxin domain amino acid sequence or a C-terminal protoxin domain amino sequence lacking 1, 2, 3, 4, 5 or more residues from the native protoxin domain C-terminal segment N- and/or C-terminus. In certain embodiments, the linker peptide and nanobody are inserted in the protoxin domain (e.g., are inserted between the N-terminal and C-terminal segments of the protoxin domain) at any one of about 80, 90, 100, or 110 to any one of about 120, 130, 140, or 150 amino acid residues C-terminal to the C-terminus of the Bt protein domain III. In certain embodiments, the linker peptide and nanobody are inserted in the protoxin domain (e.g., are inserted between the N-terminal and C-terminal segments of the protoxin domain) at any one of about 110, 111, 112, 113, or 114 to any one of about 116, 117, 118, 119, or 120 amino acid residues C-terminal to the C-terminus of the Bt protein domain III. In certain embodiments, the linker peptide and nanobody are inserted in the protoxin (e.g., are inserted between the N-terminal and C-terminal segments of the protoxin domain) at any one of about 115 amino acid residues C-terminal to the C-terminus of the Bt protein domain III. In certain embodiments, the linker peptide, nanobody, and the peptide sequence AAAYPYDVPDYGS (SEQ ID NO: 92) are inserted between amino acid residue proline 723 and amino acid residue glycine 724 in the full length Cry1Ac1Ac1F sequence of SEQ ID NO: 2. Also provided herein are polynucleotides encoding the nanobodies disclosed herein and recombinant polynucleotides comprising the same.

In certain embodiments, the composite polypeptide provided herein has at least 85%, 86%, 87%, 88%, 89%, 90%, 91%, 92%, 93%, 94%, 95%, 96%, 97%, 98%, or 99% sequence identity to SEQ ID NO: 41, 43, 45, 47, 49, 51, 53, 55, 57, 59, 61, 63, 65, 67, 69, 71, 73, 75, 77, 79, or 94. Also provided herein are polynucleotides encoding the composite polypeptides disclosed herein and recombinant polynucleotides comprising the same.

In another embodiment, the disclosure provides a polynucleotide encoding the polypeptide. In some embodiments, the polynucleotide further comprises a promoter and a terminator element which are operably linked to the polynucleotide encoding the polypeptide. The promoter and terminator can include a promoter and terminator that is functional in a cell (e.g., a bacterial cell, a plant cell, a fungal cell, an insect or other invertebrate cell, or an animal cell). In certain embodiments, the promoter and terminator are recognized by a bacterial, plant, fungal, insect, invertebrate, or animal RNA polymerase. In other embodiments, the promoter and terminator are recognized by a bacterial, plant, fungal, insect, invertebrate, or animal RNA polymerase. The promoter and terminator can be native, analogous, foreign or heterologous to the host organism and/or to the sequence of the embodiments. Additionally, the promoter can be the natural sequence or alternatively a synthetic sequence. The term “foreign” as used herein indicates that the promoter is not found in the native organism into which the promoter is introduced.

In another embodiment, cells comprising a polypeptide or polynucleotide of the present disclosure are provided. Cells comprising a polypeptide or polynucleotides include prokaryotic (e.g., a bacterium, such as a bacterium capable of transforming a eukaryotic cell) or eukaryotic (e.g., a plant cell, fungal cell, or animal cell such as an insect cell) cells. In certain embodiments, the cell is a bacterial cell. In certain embodiments, the bacterial cell is a Bacillus sp. cell, an Agrobacterium sp. cell, or a Corynebacterium cell. Bacillus sp. cells include Bacillus species capable of forming an exosporium such as Bacillus anthracis, Bacillus cereus, Bacillus thuringiensis, Bacillus mycoides, Bacillus pseudomycoides, Bacillus samanii, Bacillus gaemokensis, Bacillus weihenstephensis, and Bacillus toyoiensis as well as endophytic strains of such Bacillus species (U.S. Pat. No. 9,845,342, incorporated herein by reference in its entirety). In certain embodiments, the bacterial cell comprises a Bacillus thuringiensis (Bt) spore. In some embodiments, the Bt spore comprises a parasporal crystal comprising the polypeptide of the present disclosure.

In certain embodiments, plant, plant part, and plant cell, including both monocot and dicot plant cells, which are at least partially coated or otherwise comprise the polypeptides disclosed herein, compositions comprising the polypeptides, cells comprising the polypeptides (e.g., a Bt spore comprising a parasporal crystal comprising the polypeptide), and compositions comprising the cells) are provided. In certain embodiments, a plant, plant part, or plant cell can comprise a polypeptide disclosed herein when the polypeptide is located on the surface of the plant, plant part, or plant cell and/or when the polypeptide is located within the plant, plant part, or plant cell. Plants, plant parts, and plant cells can include plants, plant parts, and plant cells from row crop plants, fruit-producing plants and trees, vegetables, trees, and ornamental plants including ornamental flowers, shrubs, trees, groundcovers, and turf grasses. In certain embodiments, the plant, plant part, or plant cell is any of barley, corn, oat, rice, rye, sorghum, turf grass, sugarcane, wheat, alfalfa, banana, broccoli, bean, cabbage, canola, carrot, cassava, cauliflower, celery, citrus, cotton, a cucurbit, eucalyptus, flax, garlic, grape, onion, lettuce, pea, peanut, pepper, potato, poplar, pine, sunflower, safflower, soybean, strawberry, sugar beet, sweet potato, tobacco, tomato ornamental, shrub, nut, chickpea, pigeon pea, millet, hop, and pasture grasse plant, plant part, or plant cell. More specifically, a plant plant part, or plant cell according to the present disclosure can be any one of barley (Hordeum vulgare), sorghum (Sorghum bicolor), rye (Secale cereale), Triticale, sugar cane (Saccharum officinarium), maize (Zea mays), foxtail millet (Setaria italic), rice (Oryza sativa), Oryza minuta, Oryza australiensis, Oryza alta, wheat (Triticum aestivum), Triticum durum, Hordeum bulbosum, purple false brome (Brachypodium distachyon), sea barley (Hordeum marinum), goat grass (Aegilops tauschii), apple (Malus domestica), strawberry, sugar beet (Beta vulgaris), sunflower (Helianthus annuus), Australian carrot (Daucus glochidiatus), American wild carrot (Daucus pusillus), Daucus muricatus, carrot (Daucus carota), eucalyptus (Eucalyptus grandis), Erythranthe guttata, Genlisea aurea, woodland tobacco (Nicotiana sylvestris), tobacco (Nicotiana tabacum), Nicotiana tomentosiformis, tomato (Solanum lycopersicum), potato (Solanum tuberosum), coffee (Coffea canephora), grape vine (Vitis vinifera), cucumber (Cucumis sativus), mulberry (Morus notabilis), thale cress (Arabidopsis thaliana), Arabidopsis lyrata, sand rock-cress (Arabidopsis arenosa), Crucihimalaya himalaica, Crucihimalaya wallichii, wavy bittercress (Cardamine flexuosa), peppergrass (Lepidium virginicum), sheperd's-purse (Capsella bursa-pastoris), Olmarabidopsis pumila, hairy rockcress (Arabis hirsuta), rape (Brassica napus), broccoli (Brassica oleracea), Brassica rapa, Brassica juncacea, black mustard (Brassica nigra), radish (Raphanus sativus), Eruca vesicaria sativa, orange (Citrus sinensis), Jatropha curcas, cotton (Gossipium sp.), soybean (Glycine max), and black cottonwood (Populus trichocarpa). In certain embodiments, the plant, plant part, or plant cell is a barley, sorghum, rye, soybean, wheat, rice, triticale, cotton, or maize plant, plant part, or plant cell.

### Compositions

Also provided are compositions comprising insecticidal fusion polypeptides of the present disclosure as well as compositions comprising cells comprising the polypeptides (e.g., a Bt spore comprising a parasporal crystal comprising the polypeptide). In certain embodiments, the composition comprises a polypeptide of the present disclosure and at least one additional ingredient. In some embodiments, the composition comprises at least two, at least three, or at least four additional ingredients. In some embodiments, the at least one additional ingredient is a surfactant, excipient, carrier, diluent, solubilizer, adjuvant, stabilizer, sticking agent, emulsifier, desiccant, buffer, or a combination thereof. In some embodiments, the additional ingredient is an insecticide, fungicide, and/or nematicide.

In certain embodiments, the composition comprises a cell comprising a polypeptide of the present disclosure or a polynucleotide encoding a polypeptide of the present disclosure and at least one additional ingredient. In some embodiments, the composition comprises at least two, at least three, or at least four additional ingredients. In some embodiments, the at least one additional ingredient is a surfactant, excipient, carrier, diluent, solubilizer, adjuvant, stabilizer, sticking agent, emulsifier, desiccant, buffer, or a combination thereof. In some embodiments, the additional ingredient is an insecticide, fungicide, and/or nematicide. In certain embodiments, the cell is a Bacillus sp. cell, an Agrobacterium sp. cell, or a Corynebacterium cell. In some embodiments, the Bacillus sp. cell comprises a Bacillus thuringiensis (Bt) spore. In certain embodiments, the Bt spore comprises a parasporal crystal comprising the polypeptide. In certain embodiments, the Bacillus sp. is a Bacillus species capable of forming an exosporium such as Bacillus anthracis, Bacillus cereus, Bacillus thuringiensis, Bacillus mycoides, Bacillus pseudomycoides, Bacillus samanii, Bacillus gaemokensis, Bacillus weihenstephensis, and Bacillus toyoiensis or an endophytic strain of such Bacillus species (U.S. Pat. No. 9,845,342, incorporated herein by reference in its entirety).

In some embodiments, compositions of the present disclosure are formulated into a liquid, a gel, or a powder. In some embodiments, the formulation is configured to be sprayed on plants, to be injected into plants (e.g., into the vascular system of a plant), to be rubbed on leaves, to be soaked into plants, to be coated onto plants, or be coated on seeds, or to be delivered through root uptake (e.g., in a hydroponic system or via soil). In certain embodiments, the formulations can be prepared into edible “baits” or fashioned into pest “traps” to permit feeding or ingestion of the insecticidal formulation by a target pest.

Depending on the intended objectives and prevailing circumstances, the composition can be formulated into emulsifiable concentrates, suspension concentrates, directly sprayable or dilutable solutions, coatable pastes, diluted emulsions, spray powders, soluble powders, dispersible powders, wettable powders, dusts, granules, encapsulations in polymeric substances, microcapsules, foams, aerosols, carbon dioxide gas preparations, tablets, resin preparations, paper preparations, nonwoven fabric preparations, or knitted or woven fabric preparations. In some instances, the composition is a liquid. In some instances, the composition is a solid. In some instances, the composition is an aerosol, such as in a pressurized aerosol can.

In some embodiments, the composition comprises a carrier. In some embodiments the composition is an emulsion or a reverse emulsion, a liquid, or a gel. In embodiments, the composition includes a carrier that serves as a physical support (e.g., solid or semi-solid surfaces or matrices, powders, or particles or nanoparticles). In embodiments, the active agent is encapsulated or enclosed in or attached to or complexed with a carrier including a liposome, vesicle, micelle, or other fluid compartment. In embodiments, the active agent is encapsulated or enclosed in or attached to or complexed with a carrier including a naturally occurring or synthetic, branched or linear polymer (e.g., pectin, agarose, chitin, chitosan, DEAE-dextran, polyvinylpyrrolidone (“PVP”), or polyethylenimine (‘PEI”)). In embodiments, the carrier includes cations or a cationic charge, such as cationic liposomes or cationic polymers such as polyamines (e.g., spermine, spermidine, putrescine). In embodiments, the carrier includes a polypeptide such as an enzyme, (e.g., cellulase, pectolyase, maceroenzyme, pectinase), a cell penetrating or pore-forming peptide (e.g., poly-lysine, poly-arginine, or polyhomoarginine peptides).

Non-limiting examples of carriers include cationic liposomes and polymer nanoparticles reviewed by Zhang et al. (2007) J. Controlled Release, 123:1-10, and the cross-linked multilamellar liposomes described in US Patent Application Publication 2014/0356414 A1, incorporated by reference in its entirety herein. In embodiments, the carrier includes a nanomaterial, such as carbon or silica nanoparticles, carbon nanotubes, carbon nanofibers, or carbon quantum dots. Non-limiting examples of carriers include particles or nanoparticles (e.g., particles or nanoparticles made of materials such as carbon, silicon, silicon carbide, gold, tungsten, polymers, or ceramics) in various size ranges and shapes, magnetic particles or nanoparticles (e.g., silenceMag Magnetotransfection™ agent, OZ Biosciences, San Diego, CA), abrasive or scarifying agents, needles or microneedles, matrices, and grids.

In some aspects, the composition includes an excipient, e.g., a delivery vehicle, adjuvant, diluent, surfactant, stabilizer, or tonicity agent or a combination thereof. In some embodiments, the excipient is a crop oil concentrate, a vegetable oil concentrate, a modified vegetable oil, a nitrogen source, a deposition (drift control) and/or retention agent (with or without ammonium sulfate and/or defoamer), a compatibility agent, a buffering agent and/or acidifier, a water conditioning agent, a basic blend, a spreader-sticker and/or extender, an adjuvant plus foliar fertilizer, an antifoam agent, a foam marker, a scent, or a tank cleaner and/or neutralizer. In some embodiments, the excipient is an adjuvant described in the Compendium of Herbicide Adjuvants (Young et al. (2016). Compendium of Herbicide Adjuvants (13^{th} ed.), Purdue University).

Examples of delivery vehicles and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. Further examples of delivery vehicles include, but are not limited to, solid or liquid excipient materials, solvents, stabilizers, slow-release excipients, colorings, and surface-active substances (surfactants). In some instances, the excipient (e.g., delivery vehicle) is a stabilizing vehicle. In embodiments, the stabilizing vehicle includes, e.g., an epoxidized vegetable oil, an antifoaming agent, e.g. silicone oil, a preservative, a viscosity regulator, a binding agent, or a tackifier. In some instances, the stabilizing vehicle is a buffer suitable for the polypeptide. In some instances, the composition is microencapsulated in a polymer bead delivery vehicle. In some instances, the stabilizing vehicle protects the polypeptide against UV and/or acidic conditions. In some instances, the delivery vehicle contains a pH buffer. In some instances, the composition is formulated to have a pH in the range of about 4.5 to about 9.0, including for example pH ranges of about any one of 5.0 to about 8.0, about 6.5 to about 7.5, or about 6.5 to about 7.0.

In some instances, the composition provided herein includes an adjuvant. Adjuvants are agents that do not possess the polypeptide activity but impart beneficial properties to a formulation. For example, adjuvants are either pre-mixed in the formulation or added to a spray tank to improve mixing or application or to enhance performance. They are used extensively in products designed for foliar applications. Adjuvants can be used to customize the formulation to specific needs and compensate for local conditions. Adjuvants can be designed to perform specific functions, including wetting, spreading, sticking, reducing evaporation, reducing volatilization, buffering, emulsifying, dispersing, reducing spray drift, and reducing foaming. No single adjuvant can perform all these functions, but compatible adjuvants often can be combined to perform multiple functions simultaneously.

Among nonlimiting examples of adjuvants included in the formulation are binders, dispersants and stabilizers, specifically, for example, casein, gelatin, polysaccharides (e.g., starch, gum arabic, cellulose derivatives, alginic acid, etc.), lignin derivatives, bentonite, sugars, synthetic water-soluble polymers (e.g., polyvinyl alcohol, polyvinylpyrrolidone, polyacrylic acid, etc.), PAP (acidic isopropyl phosphate), BHT (2,6-di-t-butyl-4-methylphenol), BHA (a mixture of 2-t-butyl-4-methoxyphenol and 3-t-butyl-4-methoxyphenol), vegetable oils, mineral oils, fatty acids and fatty acid esters.

In embodiments, the compositions provided herein are in a liquid formulation. Liquid formulations are generally mixed with water, but in some instances are used with crop oil, diesel fuel, kerosene or other light oil as an excipient. The amount of active ingredient (e.g., polypeptides) often ranges from about 0.5 to about 80 percent by weight.

In embodiments, an emulsifiable concentrate formulation contains a liquid active ingredient, one or more petroleum-based solvents, and an agent that allows the formulation to be mixed with water to form an emulsion. Such concentrates can be used in agricultural, ornamental and turf, forestry, structural, food processing, livestock, and public health pest formulations. In embodiments, these are adaptable to application equipment from small portable sprayers to hydraulic sprayers, low-volume ground sprayers, mist blowers, and low-volume aircraft sprayers. Some active ingredients readily dissolve in a liquid excipient. When mixed with an excipient, they form a solution that does not settle out or separate, e.g., a homogenous solution. In embodiments, formulations of these types include an active ingredient, a carrier and/or an excipient, and one or more other ingredients. Solutions can be used in any type of sprayer, indoors and outdoors.

In some instances, the composition is formulated as an invert emulsion. An invert emulsion is a water-soluble active ingredient dispersed in an oil excipient. Invert emulsions require an emulsifier that allows the active ingredient to be mixed with a large volume of petroleum-based excipient, usually fuel oil. Invert emulsions aid in reducing drift. With other formulations, some spray drift results when water droplets begin to evaporate before reaching target surfaces; as a result the droplets become very small and lightweight. Because oil evaporates more slowly than water, invert emulsion droplets shrink less and more active ingredient reaches the target. Oil further helps to reduce runoff and improve rain resistance. It further serves as a sticker-spreader by improving surface coverage and absorption. Because droplets are relatively large and heavy, it is difficult to get thorough coverage on the undersides of foliage. Invert emulsions are most commonly used along rights-of-way where drift to susceptible non-target areas can be a problem.

A flowable or liquid formulation combines many of the characteristics of emulsifiable concentrates and wettable powders. Manufacturers use these formulations when the active ingredient is a solid that does not dissolve in either water or oil. The active ingredient, impregnated on a substance such as clay, is ground to a very fine powder. The powder is then suspended in a small amount of liquid. The resulting liquid product is quite thick. Flowables and liquids share many of the features of emulsifiable concentrates, and they have similar disadvantages. They require moderate agitation to keep them in suspension and leave visible residues, similar to those of wettable powders.

Flowables/liquids are easy to handle and apply. Because they are liquids, they are subject to spilling and splashing. They contain solid particles, so they contribute to abrasive wear of nozzles and pumps. Flowable and liquid suspensions settle out in their containers. Because flowable and liquid formulations tend to settle, packaging in containers of five gallons or less makes remixing easier.

Aerosol formulations contain one or more active ingredients and a solvent. Most aerosols contain a low percentage of active ingredients. There are two types of aerosol formulations—the ready-to-use type commonly available in pressurized sealed containers and those products used in electrical or gasoline-powered aerosol generators that release the formulation as a smoke or fog.

Ready to use aerosol formulations are usually small, self-contained units that release the formulation when the nozzle valve is triggered. The formulation is driven through a fine opening by an inert gas under pressure, creating fine droplets. These products are used in greenhouses, in small areas inside buildings, or in localized outdoor areas. Commercial models, which hold five to 5 pounds of active ingredient, are usually refillable.

Smoke or fog aerosol formulations are not under pressure. They are used in machines that break the liquid formulation into a fine mist or fog (aerosol) using a rapidly whirling disk or heated surface.

In some embodiments, the composition comprises a liquid excipient. In embodiments, a liquid excipient includes, for example, aromatic or aliphatic hydrocarbons (e.g., xylene, toluene, alkylnaphthalene, phenylxylylethane, kerosene, gas oil, hexane, cyclohexane, etc.), halogenated hydrocarbons (e.g., chlorobenzene, dichloromethane, dichloroethane, trichloroethane, etc.), alcohols (e.g., methanol, ethanol, isopropyl alcohol, butanol, hexanol, benzyl alcohol, ethylene glycol, etc.), ethers (e.g., diethyl ether, ethylene glycol dimethyl ether, diethylene glycol monomethyl ether, diethylene glycol monoethyl ether, propylene glycol monomethyl ether, tetrahydrofuran, dioxane, etc.), esters (e.g., ethyl acetate, butyl acetate, etc.), ketones (e.g., acetone, methyl ethyl ketone, methyl isobutyl ketone, cyclohexanone, etc.), nitriles (e.g., acetonitrile, isobutyronitrile, etc.), sulfoxides (e.g., dimethyl sulfoxide, etc.), amides (e.g., N,N-dimethylformamide, N,N-dimethylacetamide, cyclic imides (e.g. N-methylpyrrolidone) alkylidene carbonates (e.g., propylene carbonate, etc.), vegetable oil (e.g., soybean oil, cottonseed oil, etc.), vegetable essential oils (e.g., orange oil, hyssop oil, lemon oil, etc.), or water.

In some embodiments, the composition comprises a gaseous excipient. Gaseous excipients include, for example, butane gas, floron gas, liquefied petroleum gas (LPG), dimethyl ether, and carbon dioxide gas.

In some embodiments, the compositions are provided as a dry formulation. Dry formulations can be divided into two types: ready-to-use and concentrates that must be mixed with water to be applied as a spray. Most dust formulations are ready to use and contain a low percentage of active ingredients (less than about 10 percent by weight), plus a very fine, dry inert excipient (e.g., talc, chalk, clay, nut hulls, or volcanic ash). The size of individual dust particles varies. A few dust formulations are concentrates and contain a high percentage of active ingredients. In some embodiments, these are mixed with dry inert excipients before applying. In some embodiments, dusts are used dry and can easily drift to non-target sites.

In some instances, the composition is formulated as a powder. In some instances, the composition is formulated as a wettable powder. Wettable powders are dry, finely ground formulations that look like dusts. They usually must be mixed with water for application as a spray. A few products, however, can be applied either as a dust or as a wettable powder—the choice is left to the applicator. Wettable powders have about 1 to about 95 percent active ingredient by weight; in some cases more than about 50 percent. The particles do not dissolve in water. They settle out quickly unless constantly agitated to keep them suspended. They can be used for most pest problems and in most types of spray equipment where agitation is possible. Wettable powders have excellent residual activity. Because of their physical properties, most of the formulation remains on the surface of treated porous materials such as concrete, plaster, and untreated wood. In such cases, only the water penetrates the material.

In some instances, the composition is formulated as a soluble powder. Soluble powder formulations look like wettable powders. However, when mixed with water, soluble powders dissolve readily and form a true solution. After they are mixed thoroughly, no additional agitation is necessary. The amount of active ingredient in soluble powders ranges from about 15 to about 95 percent by weight; in some cases more than about 50 percent. Soluble powders have all the advantages of wettable powders and none of the disadvantages, except the inhalation hazard during mixing.

In some instances, the composition is formulated as a water-dispersible granule. Water-dispersible granules, also known as dry flowables, are like wettable powders, except instead of being dust-like, they are formulated as small, easily measured granules. Water-dispersible granules must be mixed with water to be applied. Once in water, the granules break apart into fine particles similar to wettable powders. The formulation requires constant agitation to keep it suspended in water. The percentage of active ingredient is high, often as much as 90 percent by weight. Water-dispersible granules share many of the same advantages and disadvantages of wettable powders, except they are more easily measured and mixed. Because of low dust, they cause less inhalation hazard to the applicator during handling.

In some embodiments, the composition comprises a solid excipient. Solid excipients include finely-divided powder or granules of clay (e.g. kaolin clay, diatomaceous earth, bentonite, Fubasami clay, acid clay, etc.), synthetic hydrated silicon oxide, talc, ceramics, other inorganic minerals (e.g., sericite, quartz, sulfur, activated carbon, calcium carbonate, hydrated silica, etc.), a substance which can be sublimated and is in the solid form at room temperature (e.g., 2,4,6-triisopropyl-1,3,5-trioxane, naphthalene, p-dichlorobenzene, camphor, adamantan, etc.); wool; silk; cotton; hemp; pulp; synthetic resins (e.g., polyethylene resins such as low-density polyethylene, straight low-density polyethylene and high-density polyethylene; ethylene-vinyl ester copolymers such as ethylene-vinyl acetate copolymers; ethylene-methacrylic acid ester copolymers such as ethylene-methyl methacrylate copolymers and ethylene-ethyl methacrylate copolymers; ethylene-acrylic acid ester copolymers such as ethylene-methyl acrylate copolymers and ethylene-ethyl acrylate copolymers; ethylene-vinylcarboxylic acid copolymers such as ethylene-acrylic acid copolymers; ethylene-tetracyclododecene copolymers; polypropylene resins such as propylene homopolymers and propylene-ethylene copolymers; poly-4-methylpentene-1, polybutene-1, polybutadiene, polystyrene; acrylonitrile-styrene resins; styrene elastomers such as acrylonitrile-butadiene-styrene resins, styrene-conjugated diene block copolymers, and styrene-conjugated diene block copolymer hydrides; fluororesins; acrylic resins such as poly(methyl methacrylate); polyamide resins such as nylon 6 and nylon 66; polyester resins such as polyethylene terephthalate, polyethylene naphthalate, polybutylene terephthalate, and polycyclohexylenedimethylene terephthalate; polycarbonates, polyacetals, polyacrylsulfones, polyarylates, polyacrylates, hydroxybenzoic acid polyesters, polyetherimides, polyester carbonates, polyphenylene ether resins, polyvinyl chloride, polyvinylidene chloride, polyurethane, and porous resins such as foamed polyurethane, foamed polypropylene, or foamed ethylene, etc.), glasses, metals, ceramics, fibers, cloths, knitted fabrics, sheets, papers, yarn, foam, porous substances, and multifilaments.

In some instances, the composition is provided in a microencapsulated formulation (e.g., a nanocapsule). Microencapsulated formulations are mixed with water and sprayed in the same manner as other sprayable formulations. After spraying, the plastic coating breaks down and slowly releases the active ingredient.

In some instances, the composition is provided in a liposome. In some instances, the composition is provided in a vesicle.

In some instances, a composition provided herein includes a surfactant. Surfactants, also called wetting agents and spreaders, physically alter the surface tension of a spray droplet. For a formulation to perform its function properly, a spray droplet must be able to wet the foliage and spread out evenly over a leaf Surfactants enlarge the area of formulation coverage, thereby increasing exposure to the active agent. Surfactants are particularly important when applying a formulation to waxy or hairy leaves. Without proper wetting and spreading, spray droplets often run off or fail to cover leaf surfaces adequately. Too much surfactant, however, can cause excessive runoff and reduce efficacy.

Surfactants are classified by the way they ionize or split apart into electrically charged atoms or molecules called ions. A surfactant with a negative charge is anionic. One with a positive charge is cationic, and one with no electrical charge is nonionic. Formulation activity in the presence of a nonionic surfactant can be quite different from activity in the presence of a cationic or anionic surfactant. Selecting the wrong surfactant can reduce the efficacy of a product and injure the target plant. Anionic surfactants are most effective when used with contact pesticides (pesticides that control a pest by direct contact rather than being absorbed systemically). Cationic surfactants are rarely used as stand-alone surfactants because they usually are phytotoxic.

Nonionic surfactants, often used with systemic pesticides, help sprays penetrate plant cuticles. Nonionic surfactants are compatible with most pesticides, and most EPA-registered pesticides that require a surfactant recommend a nonionic type. Adjuvants include, but are not limited to, stickers, extenders, plant penetrants, compatibility agents, buffers or pH modifiers, drift control additives, defoaming agents, and thickeners.

Among nonlimiting examples of surfactants included in the compositions described herein are alkyl sulfate ester salts, alkyl sulfonates, alkyl aryl sulfonates, alkyl aryl ethers and polyoxyethylenated products thereof, polyethylene glycol ethers, polyvalent alcohol esters and sugar alcohol derivatives. In some embodiments, the surfactant is a nonionic surfactant, a surfactant plus nitrogen source, an organo-silicone surfactant, or a high surfactant oil concentrate.

In formulations and in the use forms prepared from these formulations, the polypeptide can, in embodiments, be in a mixture with other active compounds, such as pesticidal agents (e.g., insecticides, sterilants, acaricides, nematicides, molluscicides, or fungicides, attractants, growth-regulating substances, or herbicides). As used herein, the term “pesticidal agent” refers to any substance or mixture of substances intended for preventing, destroying, repelling, or mitigating any pest. A pesticide can be a chemical substance or biological agent used against pests including insects, mollusks, pathogens, weeds, nematodes, and microbes that compete with humans for food, destroy property, spread disease, or are a nuisance. The term “pesticidal agent” further encompasses other bioactive molecules such as antibiotics, antivirals pesticides, antifungals, antihelminthics, nutrients, pollen, sucrose, and/or agents that stun or slow insect movement.

In instances where the polypeptide is applied to plants, a mixture with other known compounds, such as herbicides, fertilizers, growth regulators, safeners, semiochemicals, or else with agents for improving plant properties is also possible.

### Methods of Insect Pest Control

Methods of controlling insects are also provided. In certain embodiments, the insect is a Lepidopteran, Orthopteran, Blattarian, Thysanopteran, Hymenopteran, Dipteran, Coleopteran, and/or Hemipteran insect. In some embodiments, the Lepidopteran insect is an Spodoptera spp., a Helicoverpa spp., or an Ostrinia spp.

In certain embodiments, the method of controlling insects comprises the step of cultivating a plant that is coated or partially coated with a composition comprising a polypeptide or polynucleotide of the present disclosure in the presence of insects that feed on susceptible varieties of the plant. The method can be used with any plant. In some embodiments, the plant is a corn, cotton, or soybean plant. In certain embodiments, the insect is a Lepidopteran insect.

In certain embodiments, the method of controlling insects comprises applying a composition of the present disclosure to a plant that is susceptible to insect feeding. The method can be used with any plant. In some embodiments, the plant is a corn, cotton, or soybean plant. In certain embodiments, the insect is a Lepidopteran insect. In some embodiments, the Lepidopteran insect is an Spodoptera spp., a Helicoverpa spp., or an Ostrinia spp. In certain embodiments, the composition comprises a polypeptide of the present disclosure. In certain embodiments, the composition comprises a cell comprising a polypeptide of the disclosure, or a cell comprising a polynucleotide that encodes a polypeptide of the present disclosure.

In some embodiments, the polypeptide or cell (e.g., a Bacillus thuringiensis spore comprising the polypeptide as a parasporal crystal) is delivered at a concentration of at least 0.1 grams per acre, e.g., at least 0.1, 1, 2, 5, 10, 15, 20, 25, 30, 35, 40, 45, or 50 grams per acre. In some embodiments, a formulation comprising the less than 120 liters per acre is delivered, e.g., less than 110, 100, 90, 80, 70, 60, 50, 40, 30, 20, 15, 10, 5, or 2 liters per acre or less than 1 liter per acre.

Methods of Producing Bacillus thuringiensis Spores

Also provided are methods of producing Bacillus thuringiensis spores comprising a polypeptide of the present disclosure as a protoxin crystal. In some embodiments, the method comprises the step of culturing Bacillus thuringiensis in media which induces spore formation (e.g., a yeast extract-based media, U.S. Pat. No. 9,845,342, incorporated herein by reference in its entirety) and harvesting spores from the media.

### Embodiments

Various embodiments of the compositions, systems, and methods described herein are set forth in the following set of numbered embodiments.
1. A polypeptide comprising:
(i) from N-terminus to C-terminus, polypeptides comprising domain I (DI), domain II (DII), and domain III (DIII) of a Cry1 protein, a protease cleavage site, a linker peptide, a nanobody, and a C-terminal Cry1 protoxin domain, wherein said polypeptides are operably linked; or
(ii) from N-terminus to C-terminus, polypeptides comprising domain I (DI), domain II (DII), and domain III (DIII) of a Cry1 protein, a protease cleavage site, an N-terminal segment of a Cry1 protoxin domain, a linker peptide, a nanobody; and a C-terminal segment of a Cry1 protoxin domain, wherein said polypeptides are operably linked.
2. The polypeptide of embodiment 1, wherein the DI, DII, and DIII comprise the DI, DII, and DIII of a Cry1Aa, Cry1Ab, Cry1Ac, or Cry1Fa protein.
3. The polypeptide of embodiment 1 or 2, wherein the Cry1 protein comprises a chimeric Cry1 protein wherein DI, DII, and DIII are selected from at least a first Cry1 protein and a second Cry1 protein
4. The polypeptide of any one of embodiments 1-3, wherein the polypeptide comprises the DI and DII of Cry1Ac and the DIII of Cry1Fa.
5. The polypeptide of any one of embodiments 1-4, wherein the protease cleavage site is a serine protease or trypsin cleavage site and/or optionally wherein the linker peptide and the nanobody are inserted between the N-terminal and C-terminal segments of the protoxin domain at any one of about 80, 90, 100, or 110 to any one of about 120, 130, 140, or 150 amino acid residues C-terminal to the C-terminus of the DIII polypeptide.
6. The polypeptide of any one of embodiments 1-5, wherein the linker peptide comprises a repeat of glycine residues.
7. The polypeptide of any one of embodiments 1-6, wherein the linker peptide comprises a repeat of at least four, eight, or 12 glycine residues.
8. The polypeptide of any one of embodiments 1-7, wherein the polypeptide further comprises the peptide sequence AAAYPYDVPDYGS (SEQ ID NO: 92) between the C-terminus of the nanobody and the N-terminus of the C-terminal segment of a Cry1 protoxin domain and/or wherein the linker peptide, nanobody, and the peptide sequence of SEQ ID NO: 92 are inserted between amino acid residue proline 723 and amino acid residue glycine 724 in the full length Cry1Ac1Ac1F sequence of SEQ ID NO: 2.
9 The polypeptide of any one of embodiments 1-8, wherein the nanobody was obtained by using an insect target protein not previously identified as a Cry1 toxin receptor as an immunogen or affinity probe to isolate the nanobody.
10. The polypeptide of any one of embodiments 1-9, wherein the nanobody is obtained by using an insect nutrient amino acid transporter (NAAT) protein as an immunogen or affinity probe.
11. The polypeptide of any one of embodiments 1-10, wherein the nanobody comprises an amino acid sequence having at least 85%, 90%, 95%, or 99% sequence identity to SEQ ID NO: 5, 7, 9, 11, 13, 15, 17, 19, 21, 23, 25, 27, 29, or 96.
12. The polypeptide of any one of embodiments 1-11, wherein the polypeptide has at least 85%, 90%, 95%, or 99% sequence identity to SEQ ID NO: 41, 43, 45, 47, 49, 51, 53, 55, 57, 59, 61, 63, 65, 67, 69, 71, 73, 75, 77, 79, or 94.
13. A polynucleotide encoding the polypeptide of any one of embodiments 1 to 12.
14. The polynucleotide of embodiment 13, further comprising a promoter and a terminator element which are operable linked to the polynucleotide encoding the polypeptide.
15. The polynucleotide of embodiment 14, wherein the promoter and terminator are recognized by a bacterial RNA polymerase.
16. The polynucleotide of embodiment 14, wherein the promoter and terminator are recognized by a plant RNA polymerase.
17. A cell comprising the polynucleotide of any one of embodiments 13-16.
18. The cell of embodiment 17, wherein said cell is a bacterial or plant cell.
19. The cell of embodiment 18, wherein said bacterial cell is a Bacillus spp. cell, an Agrobacterium spp. cell, or a Corynebacterium cell.
20. The cell of embodiment 19, wherein said Bacillus spp. cell comprises a Bacillus thuringiensis (Bt) spore.
21. The cell of embodiment 20, wherein said Bt spore comprises a parasporal crystal comprising the polypeptide.
22. The cell of embodiment 18, wherein said plant cell is a monocot plant cell.
23. The cell of embodiment 18, wherein said plant cell is a dicot plant cell.
24. The cell of embodiment 18, wherein said plant cell is a barley cell, sorghum cell, rye cell, soybean cell, wheat cell, rice cell, triticale cell, cotton cell, or maize cell.
25. A plant that is coated or partially coated with a composition comprising the polypeptide of any one of embodiments 1 to 12 or a polynucleotide encoding the polypeptide.
26. The plant of embodiment 25, wherein the plant is a dicot plant, optionally wherein the dicot plant is a soybean or cotton plant.
27. The plant of embodiment 25, wherein said plant is a monocot plant and optionally wherein the plant is a corn, sorghum, or wheat plant
28. A method of controlling Lepidopteran insects comprising the step of cultivating the plant of any one of embodiments 25-27 in the presence of Lepidopteran insects which feed on susceptible varieties of the plant.
29. The method of embodiment 28, wherein the Lepidopteran insect is an Spodoptera spp., a Helicoverpa spp., or an Ostrinia spp.
30. The method of embodiment 28 or 29, wherein the plant is a corn, cotton, or soybean plant.
31. A plant part comprising the polypeptide of any one of embodiments 1-12 or a polynucleotide encoding the polypeptide.
32. The plant part of embodiment 31, wherein the plant part the part is a seed, leaf, tuber, stem, root, or boll.
33. The plant part of embodiment 31, wherein the plant part is a seed.
34. A plant part of the plant of any one of embodiments 25-27, wherein the plant part is at least partially coated with the polypeptide.
35. The plant part of embodiment 34, wherein the plant part the part is a seed, leaf, tuber, stem, root, or boll.
36. The plant part of embodiment 34, wherein the plant part is a seed.
37. A biological sample comprising the polypeptide of any one of embodiments 1 to 12 or a polynucleotide encoding the polypeptide.
38. The biological sample of embodiment 37, wherein said sample is seed meal.
39. A composition comprising the polypeptide of any one of embodiments 1-12 and at least one additional ingredient.
40. The composition of embodiment 39, wherein the at least one additional ingredient is a surfactant, excipient, carrier, diluent, solubilizer, adjuvant, stabilizer, sticking agent, emulsifier, desiccant, buffer, or a combination thereof.
41. The composition of embodiment 39, wherein the additional ingredient is an insecticide, fungicide, and/or nematicide.
42. A method of controlling Lepidopteran insects comprising applying the composition of any one of embodiments 39-41 to a plant which is susceptible to Lepidopteran insect feeding.
43. The method of embodiment 42, wherein the Lepidopteran insect is an Spodoptera spp., a Helicoverpa spp., or an Ostrinia spp.
44. The method of embodiment 42 or 43, wherein the plant is a corn, cotton, or soybean plant.
45. A composition comprising the cell of any one of embodiments 17-24 and at least one additional ingredient.
46. The composition of embodiment 45, wherein the at least one additional ingredient is a surfactant, excipient, carrier, diluent, solubilizer, adjuvant, stabilizer, sticking agent, emulsifier, desiccant, buffer, or a combination thereof.
47. The composition of embodiment 46, wherein the additional ingredient is an insecticide, fungicide, and/or nematicide.
48. The composition of any one of embodiments 45-47, wherein said cell is a Bacillus sp. cell, an Agrobacterium sp. cell, or a Corynebacterium cell.
49. The composition of embodiment 48, wherein said Bacillus sp. cell comprises a Bacillus thuringiensis (Bt) spore.
50. The composition of embodiment 49, wherein said Bt spore comprises a parasporal crystal comprising the polypeptide.
51. A method of controlling Lepidopteran insects comprising applying the composition of any one of embodiments 45-50 to a plant that is susceptible to Lepidopteran insect feeding.
52. The method of embodiment 51, wherein the Lepidopteran insect is a Spodoptera spp., a Helicoverpa spp., or an Ostrinia spp.
53. The method of embodiment 51 or 52, wherein the plant is a corn, cotton, or soybean plant.
54. A method of producing Bacillus thuringiensis spores comprising the polypeptide of any one of embodiments 1-12 as a protoxin crystal, comprising the step of culturing Bacillus thuringiensis in media which induces spore formation and harvesting spores from the media.
55. A plant or plant part that is coated, partially coated, or otherwise comprises the composition of any one of embodiments 45 to 50.

### EXAMPLES

### Example 1: Creation of Cry1 Protoxin::Nanobody Fusion Proteins

The Cry1 protoxin::nanobody fusion proteins are synthetic proteins cloned via Gibson assembly and expressed in Bt strain (4D22) which does not express any Cry toxin genes. 4D22 transformants were cultured under conditions that induced Bt sporulation and crystallization of Cry protoxin and were assessed for growth rate, sporulation and crystal formation when compared to wild-type chimera toxin transformants. Sporulated cultures were then pelleted, lyophilized for 24 h at −25 °C and samples of lyophilized cultures were compared to the chimera wild-type toxin transformants by assessing for enhanced insecticidal activity against FAW neonates in artificial diet bioassays.

The genetic constructs represent variations of fusion proteins that were designed with different combinations of linker peptides and nanobody combinations inserted at different locations within the Cry1 chimera protoxin Cry1Ac1Ac1F gene where domains I and II (DI and DII) are derived from Cry1Ac, domain III (DIII) is derived from the Cry1F protein (SEQ ID NO: 3) and the C-terminal protoxin region (required for toxin crystallization) is derived from Cry1Ac.

TABLE 1
Biological Assessment Screening of Initial Fusion Protein Constructs Relative to
Chimera Toxin

| Construct | | | | | | | |
|---|---|---|---|---|---|---|---|
| (SEQ ID | | | Fusion | Growth | Spore | Crystal | Insecticidal |
| NO:) | Nanobody^{a} | Linker | Site^{b} | Rate^{c} | Formed^{d} | Structure^{e} | Activity^{f} |
| (SEQ ID | NAAT001 | PT (SEQ | A | wild-type | yes | wild-type | − |
| NO: 40 | (SEQ ID | ID NO: 38 | | | | | |
| (DNA) 41 | NO: 4 | (DNA) 39 | | | | | |
| (protein)) | (DNA) 5 | (protein)) | | | | | |
| | (protein)) | | | | | | |
| (SEQ ID | NAAT002 | PT (SEQ | A | wild-type | yes | wild-type | + |
| NO: 42 | (SEQ ID | ID NO: 38 | | | | | |
| (DNA) 43 | NO: 6 (DNA) | (DNA) 39 | | | | | |
| (protein)) | 7 (protein)) | (protein)) | | | | | |
| (SEQ ID | NAAT029 | AEK | A | wild-type | yes | wild-type | − |
| NO: 44 | (SEQ ID | (SEQ ID | | | | | |
| (DNA) 45 | NO: 16 | NO: 30 | | | | | |
| (protein)) | (DNA) 17 | (DNA) 31 | | | | | |
| | (protein)) | (protein)) | | | | | |
| (SEQ ID | NAAT031 | PT (SEQ | A | wild-type | yes | wild-type | − |
| NO: 46 | (SEQ ID | ID NO: 38 | | | | | |
| (DNA) 47 | NO: 20 | (DNA) 39 | | | | | |
| (protein)) | (DNA) 21 | (protein)) | | | | | |
| | (protein)) | | | | | | |
| (SEQ ID | NAAT029 | G8 (SEQ | A | wild-type | yes | larger | ++ |
| NO: 52 | (SEQ ID | ID NO: 36 | | | | | |
| (DNA) 53 | NO: 16 | (DNA) 37 | | | | | |
| (protein)) | (DNA) 17 | (protein)) | | | | | |
| | (protein)) | | | | | | |
| (SEQ ID | NAAT029 | G8 (SEQ | B | slower | yes | wild-type | − |
| NO: 48 | (SEQ ID | ID NO: 36 | | | | | |
| (DNA) 49 | NO: 16 | (DNA) 37 | | | | | |
| (protein)) | (DNA) 17 | (protein)) | | | | | |
| | (protein)) | | | | | | |
| (SEQ ID | NAAT029 | N/A | C | wild-type | yes | none | − |
| NO: 50 | (SEQ ID | | | | | | |
| (DNA) 51 | NO: 16 | | | | | | |
| (protein)) | (DNA) 17 | | | | | | |
| | (protein)) | | | | | | |
| (SEQ ID | NAAT001 | G8 (SEQ | A | wild-type | yes | larger | ++ |
| NO: 54 | (SEQ ID | ID NO: 36 | | | | | |
| (DNA) 55 | NO: 4 (DNA) | (DNA) 37 | | | | | |
| (protein)) | 5 (protein)) | (protein)) | | | | | |
| (SEQ ID | NAAT002 | G8 (SEQ | A | wild-type | yes | larger | ++ |
| NO: 56 | (SEQ ID | ID NO: 36 | | | | | |
| (DNA) 57 | NO: 6 (DNA) | (DNA) 37 | | | | | |
| (protein)) | 7 (protein)) | (protein)) | | | | | |
| (SEQ ID | NAAT010 | G8 (SEQ | A | wild-type | yes | larger | ++ |
| NO: 62 | (SEQ ID | ID NO: 36 | | | | | |
| (DNA) 63 | NO: 4 | (DNA) 37 | | | | | |
| (protein)) | (DNA) 5 | (protein)) | | | | | |
| | (protein)) | | | | | | |
| (SEQ ID | NAAT031 | G8 (SEQ | A | wild-type | yes | larger | ++ |
| NO: 70 | (SEQ ID | ID NO: 36 | | | | | |
| (DNA) 71 | NO: 20 | (DNA) 37 | | | | | |
| (protein)) | (DNA) 21 | (protein)) | | | | | |
| | (protein)) | | | | | | |
| (SEQ ID | CAD005 | G8 (SEQ | A | wild-type | yes | larger | − |
| NO: 72 | (SEQ ID | ID NO: 36 | | | | | |
| (DNA) 73 | NO: 20 | (DNA) 37 | | | | | |
| (protein)) | (DNA) 21 | (protein)) | | | | | |
| | (protein)) | | | | | | |
| (SEQ ID | CAD049 | G8 (SEQ | A | wild-type | yes | larger | − |
| NO: 74 | (SEQ ID | ID NO: 36 | | | | | |
| (DNA) 75 | NO: 20 | (DNA) 37 | | | | | |
| (protein)) | (DNA) 21 | (protein)) | | | | | |
| | (protein)) | | | | | | |
| (SEQ ID | CAD076 | G8 (SEQ | A | wild-type | yes | larger | + |
| NO: 76 | (SEQ ID | ID NO: 36 | | | | | |
| (DNA) 77 | NO: 20 | (DNA) 37 | | | | | |
| (protein)) | (DNA) 21 | (protein)) | | | | | |
| | (protein)) | | | | | | |
| (SEQ ID | CAD108 | G8 (SEQ | A | wild-type | yes | larger | + |
| NO: 78 | (SEQ ID | ID NO: 36 | | | | | |
| (DNA) 79 | NO: 20 | (DNA) 37 | | | | | |
| (protein)) | (DNA) 21 | (protein)) | | | | | |
| | (protein)) | | | | | | |

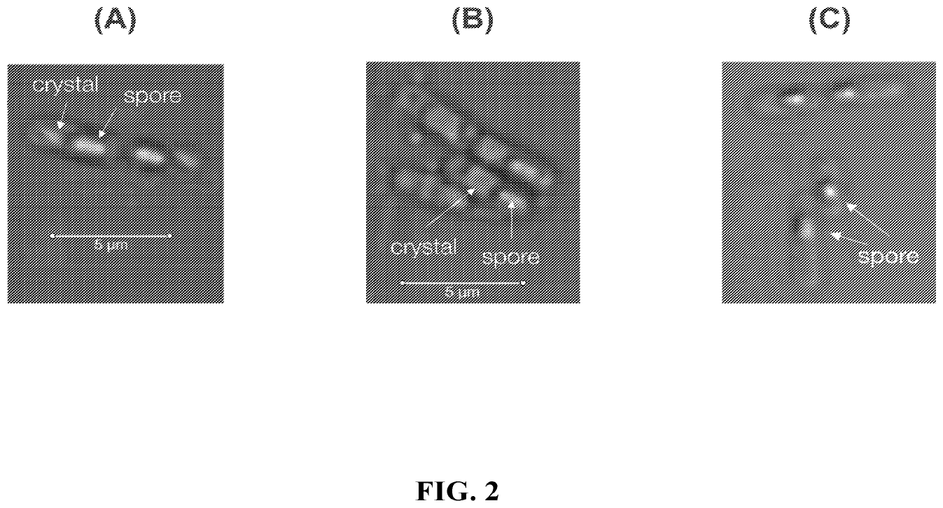

^{a}Nanobodies raised against the Spodoptera frugiperda NAAT protein are identified as “NAAT XXX.” Nanobodies raised against the Spodoptera frugiperda Cadherin protein are identified as “CAD XXX”.
^{b}Defined as (A) downstream of the serine protease site located at the C-terminus of chimera toxin DIII, (B) directly upstream of the serine protease site located at the C-terminus of chimera toxin DIII and (C) at the C-terminus of chimera DII.
^{c}Defined as the doubling time relative to chimera protoxin transformants to achieve desired bacterial concentrations during induction of sporulation after 3 days for wild-type growth and slower growth defined as requiring 6 days
^{d}Defined as the detection of spores as shown in FIG. 2A
^{e}Defined as the size of crystals relative to chimera protoxin transformants produced during sporulation and shown in FIG. 2A
^{f}Defined as “+” wild-type/chimera insect mortality, “++” higher insect mortality relative to chimera toxin, and “−” lower or undetectable mortality relative to chimera toxin where data were insufficient to calculate an LC₅₀ concentration for any of the treatment groups but were either greater than or less than wild-type measurements at multiple concentrations.

The constructs were further assessed for insecticidal activity, as presented in Table 2.

TABLE 2
Active Fusion Construct Structure and Relative Potency in Susceptible Insects

| | | | | | | Fold |
|---|---|---|---|---|---|---|
| | | | | | | enhancement |
| | | | | Wild-type | | in activity vs |
| | | | | growth | Large | wild-type |
| Construct No. | | | Fusion | rate and | Crystal | protoxin |
| (SEQ ID NO:) | Nanobody | Linker | Site^{a} | sporulation | Formation | activity^{e} |
| 1 (SEQ ID | NAAT003 | G8 (SEQ | A | + | + | 2.5 |
| NO: 58 | (SEQ ID | ID NO: | | | | |
| (DNA); SEQ | NO: 8 | 36 (DNA) | | | | |
| ID NO: 59 | (DNA) 9 | 37 | | | | |
| (protein)) | (protein)) | (protein)) | | | | |
| 2 (SEQ ID | NAAT007 | G8 (SEQ | A | + | + | 2.6 |
| NO: 60 | (SEQ ID | ID NO: | | | | |
| (DNA); SEQ | NO: 10 | 36 (DNA) | | | | |
| ID NO: 61 | (DNA) 11 | 37 | | | | |
| (protein)) | (protein)) | (protein)) | | | | |
| 3 (SEQ ID | NAAT020 | G8 (SEQ | A | + | + | 2.9 |
| NO: 64 | (SEQ ID | ID NO: | | | | |
| (DNA); SEQ | NO: 12 | 36 (DNA) | | | | |
| ID NO: 65 | (DNA) 13 | 37 | | | | |
| (protein)) | (protein)) | (protein)) | | | | |
| 4 (SEQ ID | NAAT027 | G8 (SEQ | A | + | + | 3.1 |
| NO: 66 (DNA) | (SEQ ID | ID NO: | | | | |
| 67 (protein)) | NO: 14 | 36 (DNA) | | | | |
| | (DNA) 15 | 37 | | | | |
| | (protein)) | (protein)) | | | | |
| 5 (SEQ ID | NAAT030 | G8 (SEQ | A | + | + | 5.4 |
| NO: 68 (DNA) | (SEQ ID | ID NO: | | | | |
| 69 (protein)) | NO: 19 | 36 (DNA) | | | | |
| | (DNA) 20 | 37 | | | | |
| | (protein)) | (protein)) | | | | |
| 6 (SEQ ID | NAAT031 | G8 (SEQ | A | + | + | 0.8 |
| NO: 70 (DNA) | (SEQ ID | ID NO: | | | | |
| 71 (protein)) | NO: 20 | 36 (DNA) | | | | |
| | (DNA) 21 | 37 | | | | |
| | (protein)) | (protein)) | | | | |

^{a}Nanobodies raised against the Spodoptera frugiperda NAAT protein are identified as “NAAT XXX.”
^{b}Defined as (A) downstream of the serine protease site located at the C-terminus of chimera toxin DIII.
^{c}Defined as the doubling time equivalent to chimera protoxin transformants to achieve desired bacterial concentrations during induction of sporulation after 3 days and spores as shown in FIG. 2A
^{d}Defined as the size of crystals relative to chimera protoxin transformants produced during sporulation and shown in FIG. 2A.
^{e}Defined as the relative potency when compared to predicted LC₅₀ for chimera protoxin transformants as measured by ug/cm².

Based on the results of assessing each of the fusion constructs for growth rate, sporulation, crystal formation and insecticidal activity a prototypical structure for an active fusion protein was deduced, as shown in FIG. 1. The prototypic structure comprises a specific peptide linker plus nanobody combination inserted in the genetic sequence of the Cry1 protoxin, 3′ of a predicted trypsin cleavage site located at the 3′ of the DIII coding sequence.

The next phase of the staged assessment included 26 fusion constructs that fit the prototypical design structure (FIG. 1) and differed only in the nanobody used in fusion construction. All nanobodies tested were raised against the Spodoptera frugiperda nutrient amino acid transporter protein (NAAT) and differed in their complementarity-determining region 3 (CDR3). These amino acid regions are one of three hypervariable domains that determine specific antibody binding. Each construct was assessed for bacterial growth, sporulation, and crystal formation relative to wild-type chimera toxin. The constructs were also tested for their relative potency as determined by dose response studies and determination of relative LC50 values in susceptible Spodoptera frugiperda with representative data shown in Table 2. All test samples were normalized for toxin and fusion protein concentration.

Constructs that exhibited increased potency in susceptible insects were tested in a Cry1F-resistant field isolated colony of Spodoptera frugiperda and determined to have a mutation in the ABCC2 transporter protein (Banerjee, et. al. 2017) and compared to the relative potency of Cry1F protoxin and chimera protoxin susceptibility of the resistant Spodoptera frugiperda population. Representative data are shown in Table 3.

TABLE 3
Active Fusion Construct Structure and Relative Potency
in Cry1F Resistant Spodoptera frugiperda

| Construct | | | | Fold |
|---|---|---|---|---|
| Description | | | | enhancement in |
| or Number | | | Fusion | activity vs Cry1F |
| (SEQ ID NO) | Nanobody^{a} | Linker | Site^{b} | toxin activity^{c} |
| Cry 1F (SEQ ID NO: 3 | N/A | N/A | N/A | 1.0 |
| (protein)) | | | | |
| Chimera Toxin (SEQ | N/A | N/A | N/A | 2.1 |
| ID NO: 1 (DNA); SEQ | | | | |
| ID NO: 2 (protein)) | | | | |
| 1 (SEQ ID NO: 58 | NAAT003 (SEQ | G8 (SEQ ID | A | 3.6 |
| (DNA); SEQ ID NO: | ID NO: 8 (DNA) | NO: 36 (DNA) | | |
| 59 (protein)) | 9 (protein)) | 37 (protein)) | | |
| 2 (SEQ ID NO: 60 | NAAT007 (SEQ | G8 (SEQ ID | A | 4.3 |
| (DNA); SEQ ID NO: | ID NO: 10 (DNA) | NO: 36 (DNA) | | |
| 61 (protein)) | 11 (protein)) | 37 (protein)) | | |
| 5 (SEQ ID NO: 68 | NAAT030 (SEQ | G8 (SEQ ID | A | 16.1 |
| (DNA); SEQ ID NO: | ID NO: 19 (DNA) | NO: 36 (DNA) | | |
| 69 (protein)) | 20 (protein)) | 37 (protein)) | | |

^{a}Nanobodies raised against the Spodoptera frugiperda NAAT protein are identified as “NAAT XXX.”
^{b}Defined as (A) downstream of the serine protease site located at the C-terminus of chimera toxin DIII. N/A is not applicable.
^{c}Defined as the relative potency when compared to predicted LC₅₀ for Cry1Fa protoxin measured in ug/cm².

### Example 2: Efficacy of Additional Cry1 Protoxin::Nanobody Fusion Proteins Against Susceptible Helicoverpa zea

Selected Cry1 protoxin::nanobody fusion protein constructs were tested for efficacy against susceptible Helicoverpa zea to assess insect spectrum effects. Descriptions of these fusion protein constructs and their activity against Helicoverpa zea is provided in Table 4.

TABLE 4
Active Fusion Construct Structure and Relative Potency in Helicoverpa zea

| | | | | Fold |
|---|---|---|---|---|
| | | | | enhancement |
| | | | | in activity vs |
| Construct | | | | wild-type |
| Description | | | Fusion | protoxin |
| (SEQ ID NO) | Nanobody^{a} | Linker | Site^{b} | activity^{e} |
| SEQ ID NO: 93 | NAAT018 | G8 (SEQ ID | A | 2.1 |
| (DNA); SEQ ID | (SEQ ID NO: 95 (DNA); | NO: 36 (DNA) | | |
| NO: 94 (protein)) | SEQ ID NO: 96 (protein)) | 37 (protein)) | | |
| (SEQ ID NO: 64 | NAAT020 (SEQ ID | G8 (SEQ ID | A | 4.5 |
| (DNA); SEQ ID | NO: 12 (DNA); SEQ ID | NO: 36 (DNA) | | |
| NO: 65 (protein)) | NO: 13 (protein)) | 37 (protein)) | | |
| (SEQ ID NO: 68 | NAAT030 (SEQ ID | G8 (SEQ ID | A | 3.3 |
| (DNA); SEQ ID | NO: 19 (DNA); SEQ ID | NO: 36 (DNA) | | |
| NO: 69 (protein)) | NO: 20 (protein)) | 37 (protein)) | | |
| (SEQ ID NO: 70 | NAAT031 (SEQ ID NO: | G8 (SEQ ID | A | 2.7 |
| (DNA); SEQ ID | 20 (DNA); SEQ ID NO: | NO: 36 (DNA) | | |
| NO: 71 (protein)) | 21 (protein)) | 37 (protein)) | | |
| (SEQ ID NO: 74 | CAD049 (SEQ ID NO: 20 | G8 (SEQ ID | A | 3.2 |
| (DNA); SEQ ID | (DNA); SEQ ID NO: 21 | NO: 36 (DNA) | | |
| NO: 75 (protein)) | (protein)) | 37 (protein)) | | |
| (SEQ ID NO: 76 | CAD076 (SEQ ID NO: 20 | G8 (SEQ ID | A | 3.5 |
| (DNA); SEQ ID | (DNA); SEQ ID NO: 21 | NO: 36 (DNA) | | |
| NO: 77 (protein)) | (protein)) | 37 (protein)) | | |
| (SEQ ID NO: 78 | CAD108 (SEQ ID NO: 20 | G8 (SEQ ID | A | 3.3 |
| (DNA); SEQ ID | (DNA); SEQ ID NO: 21 | NO: 36 (DNA) | | |
| NO: 79 (protein)) | (protein)) | 37 (protein)) | | |

^{a}Nanobodies raised against the Spodoptera frugiperda NAAT protein are identified as “NAAT XXX.”
^{b}Defined as (A) downstream of the serine protease site located at the C-terminus of chimera toxin DIII.
^{c} Defined as the relative potency when compared to predicted LC₅₀ for Cry1Ac1Ac1F protoxin measured in ug/cm².

The breadth and scope of the present disclosure should not be limited by any of the above-described examples.

TABLE 5
Description of Biological Sequences in Sequence
Listing

| | SEQ | |
|---|---|---|
| | ID | Sequence Type |
| Description | NO | DNA/PRT |
| Cry1Ac1Ac1F | 1 | DNA |
| Cry1Ac1Ac1F | 2 | PRT |
| Cry1Fa | 3 | PRT |
| NAAT001 Nanobody | 4 | DNA |
| NAAT001 Nanobody | 5 | PRT |
| NAAT002 Nanobody | 6 | DNA |
| NAAT002 Nanobody | 7 | PRT |
| NAAT003 Nanobody | 8 | DNA |
| NAAT003 Nanobody | 9 | PRT |
| NAAT007 Nanobody | 10 | DNA |
| NAAT007 Nanobody | 11 | PRT |
| NAAT020 Nanobody | 12 | DNA |
| NAAT020 Nanobody | 13 | PRT |
| NAAT027 Nanobody | 14 | DNA |
| NAAT027 Nanobody | 15 | PRT |
| NAAT029 Nanobody | 16 | DNA |
| NAAT029 Nanobody | 17 | PRT |
| NAAT030 Nanobody | 18 | DNA |
| NAAT030 Nanobody | 19 | PRT |
| NAAT031 Nanobody | 20 | DNA |
| NAAT031 Nanobody | 21 | PRT |
| CAD005 Nanobody | 22 | DNA |
| CAD005 Nanobody | 23 | PRT |
| CAD049 Nanobody | 24 | DNA |
| CAD049 Nanobody | 25 | PRT |
| CAD076 Nanobody | 26 | DNA |
| CAD076 Nanobody | 27 | PRT |
| CAD108 Nanobody | 28 | DNA |
| CAD108 Nanobody | 29 | PRT |
| linker AEAAAK3 | 30 | DNA |
| linker AEAAAK3 | 31 | PRT |
| linker ESGSV | 32 | DNA |
| linker ESGSV | 33 | PRT |
| linker G4S1X4 | 34 | DNA |
| linker G4S1X3 | 35 | PRT |
| linker G8 | 36 | DNA |
| linker G8 | 37 | PRT |
| linker PTPT | 38 | DNA |
| linker PTPT | 39 | PRT |
| 1Ac1Ac1F-PT-NAAT001 | 40 | DNA |
| 1Ac1Ac1F-PT-NAAT001 | 41 | PRT |
| 1Ac1Ac1F-PT-NAAT002 | 42 | DNA |
| 1Ac1Ac1F-PT-NAAT002 | 43 | PRT |
| 1AC1AC1F-AEK-NAAT029 | 44 | DNA |
| 1AC1AC1F-AEK-NAAT029 | 45 | PRT |
| 1AC1AC1F-PT-NAAT031 | 46 | DNA |
| 1AC1AC1F-PT-NAAT031 | 47 | PRT |
| 1AC1AC1F-G8-NAAT029-BCS | 48 | DNA |
| 1AC1AC1F-G8-NAAT029-BCS | 49 | PRT |
| 1AC1AC-NAAT029-1F | 50 | DNA |
| 1AC1AC-NAAT029-1F | 51 | PRT |
| 1AC1AC1F-G8-NAAT029 | 52 | DNA |
| 1AC1AC1F-G8-NAAT029 | 53 | PRT |
| 1AC1AC1F-G8-NAAT001 | 54 | DNA |
| 1AC1AC1F-G8-NAAT001 | 55 | PRT |
| 1AC1AC1F-G8-NAAT002 | 56 | DNA |
| 1AC1AC1F-G8-NAAT002 | 57 | PRT |
| 1AC1AC1F-G8-NAAT003 | 58 | DNA |
| 1AC1AC1F-G8-NAAT003 | 59 | PRT |
| 1AC1AC1F-G8-NAAT007 | 60 | DNA |
| 1AC1AC1F-G8-NAAT007 | 61 | PRT |
| 1AC1AC1F-G8-NAAT010 | 62 | DNA |
| 1AC1AC1F-G8-NAAT010 | 63 | PRT |
| 1AC1AC1F-G8-NAAT020 | 64 | DNA |
| 1AC1AC1F-G8-NAAT020 | 65 | PRT |
| 1AC1AC1F-G8-NAAT027 | 66 | DNA |
| 1AC1AC1F-G8-NAAT027 | 67 | PRT |
| 1AC1AC1F-G8-NAAT030 | 68 | DNA |
| 1AC1AC1F-G8-NAAT030 | 69 | PRT |
| 1AC1AC1F-G8-NAAT031 | 70 | DNA |
| 1AC1AC1F-G8-NAAT031 | 71 | PRT |
| 1AC1AC1F-G8-CAD005 | 72 | DNA |
| 1AC1AC1F-G8-CAD005 | 73 | PRT |
| 1AC1AC1F-G8-CAD049 | 74 | DNA |
| 1AC1AC1F-G8-CAD049 | 75 | PRT |
| 1AC1AC1F-G8-CAD076 | 76 | DNA |
| 1AC1AC1F-G8-CAD076 | 77 | PRT |
| 1AC1AC1F-G8-CAD108 | 78 | DNA |
| 1AC1AC1F-G8-CAD108 | 79 | PRT |
| Cry1Fa1 | 80 | DNA |
| Cry1Fa1/Cry1Fa2 | 81 | PRT |
| Cry1Fa3 | 82 | PRT |
| Cry1Fa4 | 83 | PRT |
| Cry1Fb1 | 84 | PRT |
| Cry1Fb2 | 85 | PRT |
| Cry1Fb3 | 86 | PRT |
| Cry1Fb4 | 87 | PRT |
| Cry1Fb5 | 88 | PRT |
| Cry1Fb6 | 89 | PRT |
| Cry1Fb7 | 90 | PRT |
| Cry1Ac1Ac1F (U.S. Pat. | 91 | PRT |
| No. 6,521,442) | | |
| HA-Tag (AAAYPYDVPDYGS) | 92 | PRT |
| 1AC1AC1F-G8-NAAT018 | 93 | DNA |
| 1AC1AC1F-G8-NAAT018 | 94 | PRT |
| NAAT018 nanobody | 95 | DNA |
| NAAT018 nanobody | 96 | PRT |

What is claimed is:

1. A polypeptide comprising:
from N-terminus to C-terminus, polypeptides comprising domain I (DI), domain II (DII), and domain III (DIII) of a Cry1 protein, a protease cleavage site, an N-terminal segment of a Cry1 protoxin domain, a linker peptide, a nanobody, and a C-terminal segment of a Cry1 protoxin domain, wherein said polypeptides are operably linked.

2. The polypeptide of claim 1, wherein the DI, DII, and DIII comprise the DI, DII, and DIII of a Cry1Aa, Cry1Ab, Cry1Ac, or Cry1Fa protein.

3. The polypeptide of claim 1, wherein the Cry1 protein comprises a chimeric Cry1 protein wherein DI, DII, and DIII are selected from at least a first Cry1 protein and a second Cry1 protein

4. The polypeptide of claim 1, wherein the polypeptide comprises the DI and DII of Cry1Ac and the DIII of Cry1Fa.

5. The polypeptide of claim 1, wherein the protease cleavage site is a serine protease cleavage site and/or optionally wherein the linker peptide and the nanobody are inserted between the N-terminal and C-terminal segments of the protoxin domain at any one of about 80, 90, 100, or 110 to any one of about 120, 130, 140, or 150 amino acid residues C-terminal to the C-terminus of the DIII polypeptide.

6. The polypeptide of claim 1, wherein the linker peptide comprises a repeat of glycine residues.

7. The polypeptide of claim 6, wherein the linker peptide comprises a repeat of at least four, eight, or 12 glycine residues.

8. The polypeptide of claim 1, wherein the polypeptide further comprises the peptide sequence AAAYPYDVPDYGS (SEQ ID NO: 92) between the C-terminus of the nanobody and the N-terminus of the C-terminal segment of a Cry1 protoxin domain and/or wherein the linker peptide, nanobody, and the peptide sequence of SEQ ID NO: 92 are inserted between amino acid residue proline 723 and amino acid residue glycine 724 in the full length Cry1Ac1Ac1F sequence of SEQ ID NO: 2.

9. The polypeptide of claim 1, wherein the nanobody was obtained by using an insect target protein not previously identified as a Cry1 toxin receptor as an immunogen or affinity probe to isolate the nanobody.

10. The polypeptide of claim 1, wherein the nanobody is obtained by using an insect nutrient amino acid transporter (NAAT) protein as an immunogen or affinity probe.

11. The polypeptide of claim 1, wherein the nanobody comprises an amino acid sequence having at least 85%, 90%, 95%, or 99% sequence identity to SEQ ID NO: 5, 7, 9, 11, 13, 15, 17, 19, 21, 23, 25, 27, 29, or 96.

12. The polypeptide of claim 1, wherein the polypeptide has at least 85%, 90%, 95%, or 99% sequence identity to SEQ ID NO: 69, 41, 43, 45, 47, 49, 51, 53, 55, 57, 59, 61, 63, 65, 67, 71, 73, 75, 77, 79, or 94.

13. A polynucleotide encoding the polypeptide of any one of claims 1 to 12.

14. The polynucleotide of claim 13, further comprising a promoter and a terminator element which are operable linked to the polynucleotide encoding the polypeptide.

15. The polynucleotide of claim 14, wherein the promoter and terminator are recognized by a bacterial RNA polymerase.

16. The polynucleotide of claim 14, wherein the promoter and terminator are recognized by a plant RNA polymerase.

17. A cell comprising the polynucleotide of claim 13.

18. The cell of claim 17, wherein said cell is a bacterial or plant cell.

19. The cell of claim 18, wherein said bacterial cell is a Bacillus sp. cell, an Agrobacterium sp. cell, or a Corynebacterium cell.

20. The cell of claim 19, wherein said Bacillus sp. cell comprises a Bacillus thuringiensis (Bt) spore.

21. The cell of claim 20, wherein said Bt spore comprises a parasporal crystal comprising the polypeptide.

22. The cell of claim 18, wherein said plant cell is a monocot plant cell.

23. The cell of claim 18, wherein said plant cell is a dicot plant cell.

24. The cell of claim 18, wherein said plant cell is a barley cell, sorghum cell, rye cell, soybean cell, wheat cell, rice cell, triticale cell, cotton cell, or maize cell.

25. A plant which is coated or partially coated with a composition comprising the polypeptide of any one of claims 1 to 12 or a polynucleotide encoding the polypeptide.

26. The plant of claim 25, wherein the plant is a dicot plant, optionally wherein the dicot plant is a soybean or cotton plant.

27. The plant of claim 25, wherein said plant is a monocot plant and optionally wherein the plant is a corn, sorghum, rice, or wheat plant.

28. A method of controlling Lepidopteran insects comprising the step of cultivating the plant of claim 25 or 26 in the presence of Lepidopteran insects which feed on susceptible varieties of the plant.

29. The method of claim 28, wherein the Lepidopteran insect is a Spodoptera sp., a Helicoverpa sp., or an Ostrinia sp.

30. The method of claim 28, wherein the plant is a corn, cotton, or soybean plant.

31. A plant part comprising or at least partially coated with the polypeptide of claim 1 or a polynucleotide encoding the polypeptide.

32. The plant part of claim 31, wherein the plant part the part is a seed, leaf, tuber, stem, root, or boll.

33. The plant part of claim 31, wherein the plant part is a seed.

34. A plant part of the plant of claim 25, wherein the plant part is at least partially coated with the composition.

35. The plant part of claim 34, wherein the plant part the part is a seed, leaf, tuber, stem, root, or boll.

36. The plant part of claim 34, wherein the plant part is a seed.

37. A biological sample comprising the polypeptide of any one of claims 1 to 12 or a polynucleotide encoding the polypeptide.

38. The biological sample of claim 37, wherein said sample is seed meal.

39. A composition comprising the polypeptide of claim 1 and at least one additional ingredient.

40. The composition of claim 39, wherein the at least one additional ingredient is a surfactant, excipient, carrier, diluent, solubilizer, adjuvant, stabilizer, sticking agent, emulsifier, desiccant, buffer, or a combination thereof.

41. The composition of claim 39, wherein the additional ingredient is an insecticide, fungicide, and/or nematicide.

42. A method of controlling Lepidopteran insects comprising applying the composition of claim 39 to a plant which is susceptible to Lepidopteran insect feeding.

43. The method of claim 42, wherein the Lepidopteran insect is an Spodoptera sp., a Helicoverpa sp., or an Ostrinia sp.

44. The method of claim 42, wherein plant is a corn, cotton, or soybean plant.

45. A composition comprising the cell of claim 17 and at least one additional ingredient.

46. The composition of claim 45, wherein the at least one additional ingredient is a surfactant, excipient, carrier, diluent, solubilizer, adjuvant, stabilizer, sticking agent, emulsifier, desiccant, buffer, or a combination thereof.

47. The composition of claim 46, wherein the additional ingredient is an insecticide, fungicide, and/or nematicide.

48. The composition of claim 45, wherein said cell is a Bacillus sp. cell, an Agrobacterium sp. cell, or a Corynebacterium cell.

49. The composition of claim 48, wherein said Bacillus sp. cell comprises a Bacillus thuringiensis (Bt) spore.

50. The composition of claim 49, wherein said Bt spore comprises a parasporal crystal comprising the polypeptide.

51. A method of controlling Lepidopteran insects comprising applying the composition of claim 45 to a plant which is susceptible to Lepidopteran insect feeding.

52. The method of claim 51, wherein the Lepidopteran insect is a Spodoptera spp., a Helicoverpa spp., or an Ostrinia spp.

53. The method of claim 51, wherein the plant is a corn, rice, cotton, or soybean plant.

54. A method of producing Bacillus thuringiensis spores comprising the polypeptide of claim 1 as a protoxin crystal, comprising the step of culturing Bacillus thuringiensis in media which induces spore formation and harvesting spores from the media.

55. A plant or plant part that is coated, partially coated, or otherwise comprises the composition of claim 50.

56. The plant or plant part of claim 55, wherein the plant or plant part is a soybean, wheat, rice, cotton, or maize plant or plant part.


SEQUENCE LISTING (96 sequences)

SEQ ID NO: 1 - DNA, 3531 bp, synthetic construct
  source 1..3531: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 2 - protein, 1177 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 3 - protein, 1174 aa, Bacillus thuringiensis
MENNIQNQCVPYNCLNNPEVEILNEERSTGRLPLDISLSLTRFLLSEFVPGVGVAFGLFDLIWGFITPSDWSLFLLQIEQLIEQRIETLERNRAITTLRGLADSYEIYIEALREWEANPNNAQLREDVRIRFANTDDALITAINNFTLTSFEIPLLSVYVQAANLHLSLLRDAVSFGQGWGLDIATVNNHYNRLINLIHRYTKHCLDTYNQGLENLRGTNTRQWARFNQFRRDLTLTVLDIVALFPNYDVRTYPIQTSSQLTREIYTSSVIEDSPVSANIPNGFNRAEFGVRPPHLMDFMNSLFVTAETVRSQTVWGGHLVSSRNTAGNRINFPSYGVFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEISGSDSWRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGTFDECYPTYLYQKIDESKLKPYTRYQLRGYIEDSQDLEIYLIRYNAKHETVNVLGTGSLWPLSVQSPIRKCGEPNRCAPHLEWNPDLDCSCRDGEKCAHHSHHFSLDIDVGCTDLNEDLDVWVIFKIKTQDGHARLGNLEFLEEKPLVGEALARVKRAEKKWRDKREKLELETNIVYKEAKESVDALFVNSQYDQLQADTNIAMIHAADKRVHRIREAYLPELSVIPGVNVDIFEELKGRIFTAFFLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTANQEEYGGAYTSRNRGYDETYGSNSSVPADYASVYEEKSYTDGRRDNPCESNRGYGDYTPLPAGYVTKELEYFPETDKVWIEIGETEGTFIVDSVELLLMEE

SEQ ID NO: 4 - DNA, 402 bp, synthetic construct
  source 1..402: mol_type = other DNA
caggtgcagctgcaggagtctgggggaggattggtgcaggctgggggctctctgagactctcctgtgcagcctctggacgcaccttcagtaggtatgccatgggctggtttcgccaggctctagggaaggagcgtgagttcgtagcaggtattaactggagtggtagtatgacatactatgcagactccgtgaagggccgattcaccatctccagagacaacgccaagaacatgctgtatctgcaaatgaacagtctgaaatctgaggacacggccgtgtattactgtgccggcgtgacggtagtaggtggtgcaccagcctttgactactggggccaggggacccaggtcaccgtctcctcagcggccgcatacccgtacgacgttccggactacggttcc

SEQ ID NO: 5 - protein, 134 aa, synthetic construct
QVQLQESGGGLVQAGGSLRLSCAASGRTFSRYAMGWFRQALGKEREFVAGINWSGSMTYYADSVKGRFTISRDNAKNMLYLQMNSLKSEDTAVYYCAGVTVVGGAPAFDYWGQGTQVTVSSAAAYPYDVPDYGS

SEQ ID NO: 6 - DNA, 377 bp, synthetic construct
  source 1..377: mol_type = other DNA
caggtgcagctgcaggagtctggaggaggagtggtgcagactgggggctccctgacactctcctgtaaagcctctagacgcaccagtggctttgccatggcctggttccgccaggctccagggatggaacgtgaatttgtagcgggcattggtcggactggggataatatccactatttagattctgtgaagggccgattcaccatctctagagataataccaagaacacgctgtctctgcaaatgaacagcctgagacctggggacacggccgtctattactgtgcagcagacgtgacaaagagtggatttatttattggggccaggggacccaggtcaccgtctcctcagcggccgcatacccgtacgacgttcc

SEQ ID NO: 7 - protein, 130 aa, synthetic construct
QVQLQESGGGVVQTGGSLTLSCKASRRTSGFAMAWFRQAPGMEREFVAGIGRTGDNIHYLDSVKGRFTISRDNTKNTLSLQMNSLRPGDTAVYYCAADVTKSGFIYWGQGTQVTVSSAAAYPYDVPDYGS

SEQ ID NO: 8 - DNA, 384 bp, synthetic construct
  source 1..384: mol_type = other DNA
caggtgcagctgcaggagtctgggggaggagtggtgcagactgggggctccctgacactctcctgtaaagcctctagacgcaccagtggctttgccatggcctggttccgccaggctccagggatggaacgtgaatttgtagcgggcattggtcggactggggataatatccactatttagattctgtgaagggccgattcgccatctctagagataataccaagaacacgctgtatctgcaaatgaataacctgcaacctgaggacacggccgtctattactgttgcctcgccaaggcggcggacccgttctgcgaccagggtacccaggtcaccgtctcctcagcggccgcatacccgtacgacgttccggactacggttcc

SEQ ID NO: 9 - protein, 128 aa, synthetic construct
QVQLQESGGGVVQTGGSLTLSCKASRRTSGFAMAWFRQAPGMEREFVAGIGRTGDNIHYLDSVKGRFAISRDNTKNTLYLQMNNLQPEDTAVYYCCLAKAADPFCDQGTQVTVSSAAAYPYDVPDYGS

SEQ ID NO: 10 - DNA, 390 bp, synthetic construct
  source 1..390: mol_type = other DNA
caggtgcagctgcaggagtctgggggaggcttggtgcagcctggggggtctctgagactctcctgtgcagcctctggaagcatcttcagtatccctgccatgggctggtaccgtcaggctccagggaagcagcgcgagttggtcgcagttattactagagatggtagcacgcactatgcagactccgtgaagggccgattcaccatctccagagacaacgccaaaaacacgctgtatctgcaaatgaacagtctgaaatctgaggacacggccgtgtattactgtgccaaactgggtactagccgatcgtatgactactggggccaggggacccaggtcaccgtctcctcagcggccgcatacccgtacgacgttccggactacggttcc

SEQ ID NO: 11 - protein, 130 aa, synthetic construct
QVQLQESGGGLVQPGGSLRLSCAASGSIFSIPAMGWYRQAPGKQRELVAVITRDGSTHYADSVKGRFTISRDNAKNTLYLQMNSLKSEDTAVYYCAKLGTSRSYDYWGQGTQVTVSSAAAYPYDVPDYGS

SEQ ID NO: 12 - DNA, 414 bp, synthetic construct
  source 1..414: mol_type = other DNA
caggtgcagctgcaggagtctgggggaggattggtgcaggctgggggctctctgagactctcctgtgcagcctctaggcgcaccttcagtaactatgccatgggctggttccgccaggctccagggaaggagcgagagtttgtagcagctattagctcaagtggtggtcgcacacactatgccgactccgtgaagggccgattcaccatctccagagacaacgccaagaacacgctgtatctgcaaatggacagtctgagatctgatgacacggccgtgtattattgtgcaagggagagcgaggcttactatagcaactcaccttactacggcatggactactggggcaaagggacccaggtcaccgtctcctcagcggccgcatacccgtacgacgttccggactacggttcc

SEQ ID NO: 13 - protein, 138 aa, synthetic construct
QVQLQESGGGLVQAGGSLRLSCAASRRTFSNYAMGWFRQAPGKEREFVAAISSSGGRTHYADSVKGRFTISRDNAKNTLYLQMDSLRSDDTAVYYCARESEAYYSNSPYYGMDYWGKGTQVTVSSAAAYPYDVPDYGS

SEQ ID NO: 14 - DNA, 402 bp, synthetic construct
  source 1..402: mol_type = other DNA
caggtgcagctgcaggagtctgggggaggattggtgcaggctgggggctctctgagactctcctgtgcagcctctggacgctccttcagtacatatgccatgggctggttccgccaggctccagggaaggagcgtacatttatagcaggtatcacccggtacggcggtacatattatgcagattccgtgaaggaccgatttaccatctccagagacaacgccaagaacatagtgtatctgcaaatgaacgccctgaaacctgaggacacggccgtttattactgtgcagcacgtcccgtatatagggatagcggctattcgtatgactactggggccaggggacccaggtcaccgtctcctcagcggccgcatacccgtacgacgttccggactacggttcc

SEQ ID NO: 15 - protein, 134 aa, synthetic construct
QVQLQESGGGLVQAGGSLRLSCAASGRSFSTYAMGWFRQAPGKERTFIAGITRYGGTYYADSVKDRFTISRDNAKNIVYLQMNALKPEDTAVYYCAARPVYRDSGYSYDYWGQGTQVTVSSAAAYPYDVPDYGS

SEQ ID NO: 16 - DNA, 374 bp, synthetic construct
  source 1..374: mol_type = other DNA
caggtgcagctgcaggagtctggaggaggagtggtgcagactgggggctccctgacactctcctgtaaagcctctagacgcaccagtggctttgccatggcctggttccgccaggctccagggatggaacgtgaatttgtagcgggcattggtcggactggggataatatccactatttagattctgtgaagggccgattcaccatctctagagataataccaagaacacgctgtctctgcaaatgaacaacctgaaacctgaggacacggccgtgtattactgcctacgtacgatgggtggtacctggtctgagaagggccaggggacccaggtcaccgtctcctcagcggccgcatacccgtacgacgttcc

SEQ ID NO: 17 - protein, 129 aa, synthetic construct
QVQLQESGGGVVQTGGSLTLSCKASRRTSGFAMAWFRQAPGMEREFVAGIGRTGDNIHYLDSVKGRFTISRDNTKNTLSLQMNNLKPEDTAVYYCLRTMGGTWSEKGQGTQVTVSSAAAYPYDVPDYGS

SEQ ID NO: 18 - DNA, 402 bp, synthetic construct
  source 1..402: mol_type = other DNA
caggtgcagctgcaggagtctgggggaggagtggtgcagactgggggctccctgacactctcctgtaaagcctctagacgcaccagtggctttgccatggcctggttccgccaggctccagggatggaacgtgaatttgtagcgggcattggtcggactggggataatatccactatttagattctgtgaagggccgattcaccatctctagagataataccaagaacacgctgtctctgcaaatgaacaacctgagacgggaagatacggccaagtattactgtgcagcagtaaaatttcccaatagtgacaaccttgaatggtattactggggccaggggacccaggtcaccgtctcctcagcggccgcatacccgtacgacgttccggactacggttcc

SEQ ID NO: 19 - protein, 134 aa, synthetic construct
QVQLQESGGGVVQTGGSLTLSCKASRRTSGFAMAWFRQAPGMEREFVAGIGRTGDNIHYLDSVKGRFTISRDNTKNTLSLQMNNLRREDTAKYYCAAVKFPNSDNLEWYYWGQGTQVTVSSAAAYPYDVPDYGS

SEQ ID NO: 20 - DNA, 388 bp, synthetic construct
  source 1..388: mol_type = other DNA
caggtgcagctgcaggagtctgggggaggattggtgcaaactggggactctctgagtctctcctgtgcagcctcgggacgcaccgtcaataactatcccatggcctggttccggcaggctccaggaaaggagcgagaatttctggcaggtattagtcggaatggtgcaactacagcctatgaagactccgtgaagggccgattcaccatctccagagacaacgccaagaacacgctgtatctgcaaatgaacagtctgaaatctgaagacacggccgtctattactgtgcagcagaccctcccacttggacatactggggccaggggacccaggtcaccgtctcctcagcggccgcatacccgtacgacgttccggactacggttccc

SEQ ID NO: 21 - protein, 129 aa, synthetic construct
QVQLQESGGGLVQTGDSLSLSCAASGRTVNNYPMAWFRQAPGKEREFLAGISRNGATTAYEDSVKGRFTISRDNAKNTLYLQMNSLKSEDTAVYYCAADPPTWTYWGQGTQVTVSSAAAYPYDVPDYGS

SEQ ID NO: 22 - DNA, 387 bp, synthetic construct
  source 1..387: mol_type = other DNA
caggtgcagctgcaggagtctgggggaggctcggtgcaggctggggggtctctgaaactctcctgtgcagcttctggaaccgaaagcattttcatcgtgatgggctggtaccgccaggctccagggaaagagcgcgagttggtcgcggcaatgtcatttggtggtggtacaaatgttacagacggcgtgaggggccgattcaccatctccagagactttgacaagaacacggtggatctgcaaatgaacaacctacagactgaggacacggccgtctattactgtaatgcagtccggtggggccctcgtgactactggggccaggggacccaggtcaccgtctcctcagcggccgcatacccgtacgacgttccggactacggttcc

SEQ ID NO: 23 - protein, 129 aa, synthetic construct
QVQLQESGGGSVQAGGSLKLSCAASGTESIFIVMGWYRQAPGKERELVAAMSFGGGTNVTDGVRGRFTISRDFDKNTVDLQMNNLQTEDTAVYYCNAVRWGPRDYWGQGTQVTVSSAAAYPYDVPDYGS

SEQ ID NO: 24 - DNA, 426 bp, synthetic construct
  source 1..426: mol_type = other DNA
caggtgcagctgcaggagtctgggggaggattggtgcaggctggggcctctctgagactctcctgtgcagcctctagacgcaccggcagtagtcttaccatgggctggttccgccaggctccagggaaggagcgtgagtttgtagcagctattagccggagtggtattagaacatactacgcagactttgtgaagggccggttcaccatctccagagacaacgccaagaacacgctctatctgcaaatgaacagcctgaaacctggggacacggccgtgtattactgtgcggctaacgacaaaacatacggtagtggtcttgacgtctacacaaggcgacaagactattactactggggcccggggacccaggtcaccgtctcctcagcggccgcatacccgtacgacgttccggactacggttcc

SEQ ID NO: 25 - protein, 142 aa, synthetic construct
QVQLQESGGGLVQAGASLRLSCAASRRTGSSLTMGWFRQAPGKEREFVAAISRSGIRTYYADFVKGRFTISRDNAKNTLYLQMNSLKPGDTAVYYCAANDKTYGSGLDVYTRRQDYYYWGPGTQVTVSSAAAYPYDVPDYGS

SEQ ID NO: 26 - DNA, 426 bp, synthetic construct
  source 1..426: mol_type = other DNA
caggtgcagctgcaggagtctggaggaggattggtgcaggctgggggctctctgagactctcctgtgcagcctctagacgcctcggcagtagttatgccatggcctggttccgccaggctccagggaaggagcgtgagtttgtaacagctattagccggagtggtggcagaacatactacgcagactttgtgaagggccagttcaccatctccagagacaacgccaagaacacgctgcatctgcaaatgaacagcctgaaacctgaggacacggccgtgtattactgtgcggctaacgacaaagcatacggtaccggtcttgacgtctacacaaggcgacaagactattactactggggcccggggacccaggtcaccgtctcctcagcggccgcatacccgtacgacgttccggactacggttcc

SEQ ID NO: 27 - protein, 142 aa, synthetic construct
QVQLQESGGGLVQAGGSLRLSCAASRRLGSSYAMAWFRQAPGKEREFVTAISRSGGRTYYADFVKGQFTISRDNAKNTLHLQMNSLKPEDTAVYYCAANDKAYGTGLDVYTRRQDYYYWGPGTQVTVSSAAAYPYDVPDYGS

SEQ ID NO: 28 - DNA, 426 bp, synthetic construct
  source 1..426: mol_type = other DNA
caggtgcagctgcaggagtctgggggaggattggtgcaggctggggcctctctgagactctcctgtgcagcctctagacgcaccggcagtagtcttaccatgggctggttccgccaggctccagggaaggagcgtgagtttgtagcagctattagccggagtggtattagaacatactacgcagactttgtgaagggccggttcaccatctccagagacaacgccaagaacacgctctatctgcaaatgaacagcctgaaacctgaggacacggccgtgtattactgtgcggctaacgacaaaacatacggtagtggttttgacgtctacacaaggcgacaagactattactactggggcccggggacccaggtcaccgtctcctcagcggccgcatacccgtacgacgttccggactacggttcc

SEQ ID NO: 29 - protein, 142 aa, synthetic construct
QVQLQESGGGLVQAGASLRLSCAASRRTGSSLTMGWFRQAPGKEREFVAAISRSGIRTYYADFVKGRFTISRDNAKNTLYLQMNSLKPEDTAVYYCAANDKTYGSGFDVYTRRQDYYYWGPGTQVTVSSAAAYPYDVPDYGS

SEQ ID NO: 30 - DNA, 51 bp, synthetic construct
  source 1..51: mol_type = other DNA
gcggaggccgctgcaaaggaagctgctgcgaaggaagcagcggccaaggcg

SEQ ID NO: 31 - protein, 17 aa, synthetic construct
AEAAAKEAAAKEAAAKA

SEQ ID NO: 32 - DNA, 51 bp, synthetic construct
  source 1..51: mol_type = other DNA
gaaagcggcagcgtgagcagcgaacagctggcgcagtttcgcagcctggat

SEQ ID NO: 33 - protein, 17 aa, synthetic construct
ESGSVSSEQLAQFRSLD

SEQ ID NO: 34 - DNA, 45 bp, synthetic construct
  source 1..45: mol_type = other DNA
ggtggcggcgggagtggcgggggtggtagtggcggtgggggctcc

SEQ ID NO: 35 - protein, 15 aa, synthetic construct
GGGGSGGGGSGGGGS

SEQ ID NO: 36 - DNA, 24 bp, synthetic construct
  source 1..24: mol_type = other DNA
ggtggcggcgggggcgggggtggt

SEQ ID NO: 37 - protein, 8 aa, synthetic construct
GGGGGGGG

SEQ ID NO: 38 - DNA, 36 bp, synthetic construct
  source 1..36: mol_type = other DNA
ccgactccgactactcccacacctaccacgcccacc

SEQ ID NO: 39 - protein, 12 aa, synthetic construct
PTPTTPTPTTPT

SEQ ID NO: 40 - DNA, 3930 bp, synthetic construct
  source 1..3930: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaccgacgcctactaccccgactccgaccactcctactcaggtgcagcttcaggagtcaggtggaggtcttgttcaggctggtggatctcttcgcttgtcttgcgctgcgtcgggtcgtacattctcgcgctacgcgatgggctggttccgtcaggctttgggtaaagagcgcgagtttgtggcagggattaactggtccgggtctatgacgtactacgcagattctgttaaaggtcgcttcactatttctcgtgataacgcgaaaaacatgttgtatctgcaaatgaattctcttaaatcggaggataccgcagtttactattgcgcaggagtcaccgtcgtcggaggtgcgccagcttttgactattggggtcaaggcactcaagtaaccgtctcatcaggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 41 - protein, 1310 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPPTPTTPTPTTPTQVQLQESGGGLVQAGGSLRLSCAASGRTFSRYAMGWFRQALGKEREFVAGINWSGSMTYYADSVKGRFTISRDNAKNMLYLQMNSLKSEDTAVYYCAGVTVVGGAPAFDYWGQGTQVTVSSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 42 - DNA, 3918 bp, synthetic construct
  source 1..3918: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaccgactccgactactcccacacctaccacgcccacccaggtacaattacaggaatcagggggtggtgtagttcaaacggggggctcattgactctttcttgcaaagcgtctcgccgtacctccggttttgcgatggcctggttccgccaagcaccagggatggaacgtgaatttgttgccggtattggtcgcactggtgataacatccactaccttgactcggtgaagggtcgtttcaccatttcccgcgacaacaccaaaaataccttatccttgcaaatgaattctcttcgtccaggtgataccgccgtttattactgtgctgctgatgtcactaagtcgggcttcatctactggggtcagggcacacaggtcaccgtttcatcaggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 43 - protein, 1306 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPPTPTTPTPTTPTQVQLQESGGGVVQTGGSLTLSCKASRRTSGFAMAWFRQAPGMEREFVAGIGRTGDNIHYLDSVKGRFTISRDNTKNTLSLQMNSLRPGDTAVYYCAADVTKSGFIYWGQGTQVTVSSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 44 - DNA, 3930 bp, synthetic construct
  source 1..3930: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccagcggaggccgctgcaaaggaagctgctgcgaaggaagcagcggccaaggcgcaggtgcagctgcaggagtctggaggaggagtggtgcagactgggggctccctgacactctcctgtaaagcctctagacgcaccagtggctttgccatggcctggttccgccaggctccagggatggaacgtgaatttgtagcgggcattggtcggactggggataatatccactatttagattctgtgaagggccgattcaccatctctagagataataccaagaacacgctgtctctgcaaatgaacaacctgaaacctgaggacacggccgtgtattactgcctacgtacgatgggtggtacctggtctgagaagggccaggggacccaggtcaccgtctcctcaggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 45 - protein, 1310 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPAEAAAKEAAAKEAAAKAQVQLQESGGGVVQTGGSLTLSCKASRRTSGFAMAWFRQAPGMEREFVAGIGRTGDNIHYLDSVKGRFTISRDNTKNTLSLQMNNLKPEDTAVYYCLRTMGGTWSEKGQGTQVTVSSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 46 - DNA, 3915 bp, synthetic construct
  source 1..3915: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaccaacccctacaaccccgaccccgactacgccgactcaagttcaattacaagaaagcggaggaggtctggtccagacaggtgattcgttaagccttagttgcgctgcgagcggtcggactgttaacaattaccctatggcatggtttcgtcaagccccgggtaaagaacgggagttcctcgccggaatctcccgtaacggcgctacaacggcatacgaggattcagttaaaggtcggtttacaatttcacgcgataatgctaagaataccctctacctgcagatgaattctcttaagtcagaggatacagcagtttactattgcgctgctgatcccccaacctggacttattggggtcaaggtactcaagttactgtatcatctggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 47 - protein, 1305 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPPTPTTPTPTTPTQVQLQESGGGLVQTGDSLSLSCAASGRTVNNYPMAWFRQAPGKEREFLAGISRNGATTAYEDSVKGRFTISRDNAKNTLYLQMNSLKSEDTAVYYCAADPPTWTYWGQGTQVTVSSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 48 - DNA, 3903 bp, synthetic construct
  source 1..3903: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccaggtggcggcgggggcgggggtggtcaggtgcagctgcaggagtctggaggaggagtggtgcagactgggggctccctgacactctcctgtaaagcctctagacgcaccagtggctttgccatggcctggttccgccaggctccagggatggaacgtgaatttgtagcgggcattggtcggactggggataatatccactatttagattctgtgaagggccgattcaccatctctagagataataccaagaacacgctgtctctgcaaatgaacaacctgaaacctgaggacacggccgtgtattactgcctacgtacgatgggtggtacctggtctgagaagggccaggggacccaggtcaccgtctcctcagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 49 - protein, 1301 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPGGGGGGGGQVQLQESGGGVVQTGGSLTLSCKASRRTSGFAMAWFRQAPGMEREFVAGIGRTGDNIHYLDSVKGRFTISRDNTKNTLSLQMNNLKPEDTAVYYCLRTMGGTWSEKGQGTQVTVSSVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 50 - DNA, 3879 bp, synthetic construct
  source 1..3879: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggcaggtgcagctgcaggagtctggaggaggagtggtgcagactgggggctccctgacactctcctgtaaagcctctagacgcaccagtggctttgccatggcctggttccgccaggctccagggatggaacgtgaatttgtagcgggcattggtcggactggggataatatccactatttagattctgtgaagggccgattcaccatctctagagataataccaagaacacgctgtctctgcaaatgaacaacctgaaacctgaggacacggccgtgtattactgcctacgtacgatgggtggtacctggtctgagaagggccaggggacccaggtcaccgtctcctcaattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 51 - protein, 1293 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERQVQLQESGGGVVQTGGSLTLSCKASRRTSGFAMAWFRQAPGMEREFVAGIGRTGDNIHYLDSVKGRFTISRDNTKNTLSLQMNNLKPEDTAVYYCLRTMGGTWSEKGQGTQVTVSSITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 52 - DNA, 3903 bp, synthetic construct
  source 1..3903: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtggcggcgggggcgggggtggtcaggtgcagctgcaggagtctggaggaggagtggtgcagactgggggctccctgacactctcctgtaaagcctctagacgcaccagtggctttgccatggcctggttccgccaggctccagggatggaacgtgaatttgtagcgggcattggtcggactggggataatatccactatttagattctgtgaagggccgattcaccatctctagagataataccaagaacacgctgtctctgcaaatgaacaacctgaaacctgaggacacggccgtgtattactgcctacgtacgatgggtggtacctggtctgagaagggccaggggacccaggtcaccgtctcctcaggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 53 - protein, 1301 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGGGGGGGGQVQLQESGGGVVQTGGSLTLSCKASRRTSGFAMAWFRQAPGMEREFVAGIGRTGDNIHYLDSVKGRFTISRDNTKNTLSLQMNNLKPEDTAVYYCLRTMGGTWSEKGQGTQVTVSSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 54 - DNA, 3918 bp, synthetic construct
  source 1..3918: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtggcggcgggggcgggggtggtcaggtgcagcttcaggagtcaggtggaggtcttgttcaggctggtggatctcttcgcttgtcttgcgctgcgtcgggtcgtacattctcgcgctacgcgatgggctggttccgtcaggctttgggtaaagagcgcgagtttgtggcagggattaactggtccgggtctatgacgtactacgcagattctgttaaaggtcgcttcactatttctcgtgataacgcgaaaaacatgttgtatctgcaaatgaattctcttaaatcggaggataccgcagtttactattgcgcaggagtcaccgtcgtcggaggtgcgccagcttttgactattggggtcaaggcactcaagtaaccgtctcatcaggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 55 - protein, 1316 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGGGGGGGGQVQLQESGGGLVQAGGSLRLSCVASALSFSTHTMGWFRQAPGKDREFVAAITWNGGNTYYESSVKGRFTISRDNAKNTLYLQMNSLKSEDTAVYYCAGWVAGNPYDYWGQGTQVTVSSAAAYPYDVPDYGSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 56 - DNA, 3906 bp, synthetic construct
  source 1..3906: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtggcggcgggggcgggggtggtcaggtacaattacaggaatcagggggtggtgtagttcaaacggggggctcattgactctttcttgcaaagcgtctcgccgtacctccggttttgcgatggcctggttccgccaagcaccagggatggaacgtgaatttgttgccggtattggtcgcactggtgataacatccactaccttgactcggtgaagggtcgtttcaccatttcccgcgacaacaccaaaaataccttatccttgcaaatgaattctcttcgtccaggtgataccgccgtttattactgtgctgctgatgtcactaagtcgggcttcatctactggggtcagggcacacaggtcaccgtttcatcaggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 57 - protein, 1302 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGGGGGGGGQVQLQESGGGVVQTGGSLTLSCKASRRTSGFAMAWFRQAPGMEREFVAGIGRTGDNIHYLDSVKGRFTISRDNTKNTLSLQMNSLRPGDTAVYYCAADVTKSGFIYWGQGTQVTVSSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 58 - DNA, 3939 bp, synthetic construct
  source 1..3939: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtggcggcgggggcgggggtggtcaggtgcagctgcaggagtctgggggaggagtggtgcagactgggggctccctgacactctcctgtaaagcctctagacgcaccagtggctttgccatggcctggttccgccaggctccagggatggaacgtgaatttgtagcgggcattggtcggactggggataatatccactatttagattctgtgaagggccgattcgccatctctagagataataccaagaacacgctgtatctgcaaatgaataacctgcaacctgaggacacggccgtctattactgttgcctcgccaaggcggcggacccgttctgcgaccagggtacccaggtcaccgtctcctcagcggccgcatacccgtacgacgttccggactacggttccggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 59 - protein, 1313 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGGGGGGGGQVQLQESGGGVVQTGGSLTLSCKASRRTSGFAMAWFRQAPGMEREFVAGIGRTGDNIHYLDSVKGRFAISRDNTKNTLYLQMNNLQPEDTAVYYCCLAKAADPFCDQGTQVTVSSAAAYPYDVPDYGSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 60 - DNA, 3954 bp, synthetic construct
  source 1..3954: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtggcggcgggggcgggggtggtcaggtgcagctgcaggagtctgggggaggcttggtgcaagctggggggtctgtgagactctcctgtgtagtctctggaatcagttacgatgaatatgccatagcctggttccgccaggcccctggaaaggagcgtgagggggtctcagttatcgcgaccagtgatggtagtacatactatgcagactccgtgaagggccgattcaccatctcgcgtgacagcgacaagaacacggtgtatctgcaaatgaacagcctgaaacctgaggacacggccgtctattactgtaatgcaggggcggaagtggctggtatattacgtagctactggggccaggggacccaggtcaccgtctcctcagcggccgcatacccgtacgacgttccggactacggttccggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 61 - protein, 1323 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGGGGGGGGQVQLQESGGGLVQAGGSLRLSCAASRRTFSNYAMGWFRQAPGKEREFVAAISSSGGRTHYADSVKGRFTISRDNAKNTLYLQMDSLRSDDTAVYYCARESEAYYSNSPYYGMDYWGKGTQVTVSSAAAYPYDVPDYGSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 62 - DNA, 3912 bp, synthetic construct
  source 1..3912: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtggcggcgggggcgggggtggtcaagtgcagttacaggaatcaggtggtggactggtccaagcaggcgggtcccttacgctttcatgtgcgttcagcggacgtacgtttacacactatgctatggcgtggttccgccaagcacctgggaaagaacgtaaattcgtagcaggggtaacccgctcaggaccgaatacttattatgatgactccgtccaagggcgtttcactatcagccgtgataatgcaaagaatactgtgtaccttcacatgaattccttgaagcctgaagacacggcggtatattattgcgctgccaattccggtgtggtttctggatatgattactgggggcaaggcacgcaagtcacggtgtcgtcaggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 63 - protein, 1304 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGGGGGGGGQVQLQESGGGLVQAGGSLTLSCAFSGRTFTHYAMAWFRQAPGKERKFVAGVTRSGPNTYYDDSVQGRFTISRDNAKNTVYLHMNSLKPEDTAVYYCAANSGVVSGYDYWGQGTQVTVSSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 64 - DNA, 3969 bp, synthetic construct
  source 1..3969: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtggcggcgggggcgggggtggtcaggtgcagctgcaggagtctgggggaggattggtgcaggctgggggctctctgagactctcctgtgcagcctctaggcgcaccttcagtaactatgccatgggctggttccgccaggctccagggaaggagcgagagtttgtagcagctattagctcaagtggtggtcgcacacactatgccgactccgtgaagggccgattcaccatctccagagacaacgccaagaacacgctgtatctgcaaatggacagtctgagatctgatgacacggccgtgtattattgtgcaagggagagcgaggcttactatagcaactcaccttactacggcatggactactggggcaaagggacccaggtcaccgtctcctcagcggccgcatacccgtacgacgttccggactacggttccggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 65 - protein, 1323 aa, synthetic construct; identical to SEQ ID NO: 61

SEQ ID NO: 66 - DNA, 3957 bp, synthetic construct
  source 1..3957: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtggcggcgggggcgggggtggtcaggtgcagctgcaggagtctgggggaggattggtgcaggctgggggctctctgagactctcctgtgcagcctctggacgctccttcagtacatatgccatgggctggttccgccaggctccagggaaggagcgtacatttatagcaggtatcacccggtacggcggtacatattatgcagattccgtgaaggaccgatttaccatctccagagacaacgccaagaacatagtgtatctgcaaatgaacgccctgaaacctgaggacacggccgtttattactgtgcagcacgtcccgtatatagggatagcggctattcgtatgactactggggccaggggacccaggtcaccgtctcctcagcggccgcatacccgtacgacgttccggactacggttccggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 67 - protein, 1319 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGGGGGGGGQVQLQESGGGLVQAGGSLRLSCAASGRSFSTYAMGWFRQAPGKERTFIAGITRYGGTYYADSVKDRFTISRDNAKNIVYLQMNALKPEDTAVYYCAARPVYRDSGYSYDYWGQGTQVTVSSAAAYPYDVPDYGSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 68 - DNA, 3957 bp, synthetic construct
  source 1..3957: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtggcggcgggggcgggggtggtcaggtgcagctgcaggagtctgggggaggagtggtgcagactgggggctccctgacactctcctgtaaagcctctagacgcaccagtggctttgccatggcctggttccgccaggctccagggatggaacgtgaatttgtagcgggcattggtcggactggggataatatccactatttagattctgtgaagggccgattcaccatctctagagataataccaagaacacgctgtctctgcaaatgaacaacctgagacgggaagatacggccaagtattactgtgcagcagtaaaatttcccaatagtgacaaccttgaatggtattactggggccaggggacccaggtcaccgtctcctcagcggccgcatacccgtacgacgttccggactacggttccggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 69 - protein, 1319 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGGGGGGGGQVQLQESGGGVVQTGGSLTLSCKASRRTSGFAMAWFRQAPGMEREFVAGIGRTGDNIHYLDSVKGRFTISRDNTKNTLSLQMNNLRREDTAKYYCAAVKFPNSDNLEWYYWGQGTQVTVSSAAAYPYDVPDYGSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 70 - DNA, 3903 bp, synthetic construct
  source 1..3903: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtggcggcgggggcgggggtggtcaagttcaattacaagaaagcggaggaggtctggtccagacaggtgattcgttaagccttagttgcgctgcgagcggtcggactgttaacaattaccctatggcatggtttcgtcaagccccgggtaaagaacgggagttcctcgccggaatctcccgtaacggcgctacaacggcatacgaggattcagttaaaggtcggtttacaatttcacgcgataatgctaagaataccctctacctgcagatgaattctcttaagtcagaggatacagcagtttactattgcgctgctgatcccccaacctggacttattggggtcaaggtactcaagttactgtatcatctggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 71 - protein, 1301 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGGGGGGGGQVQLQESGGGLVQTGDSLSLSCAASGRTVNNYPMAWFRQAPGKEREFLAGISRNGATTAYEDSVKGRFTISRDNAKNTLYLQMNSLKSEDTAVYYCAADPPTWTYWGQGTQVTVSSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 72 - DNA, 3942 bp, synthetic construct
  source 1..3942: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtggcggcgggggcgggggtggtcaggtgcagctgcaggagtctgggggaggctcggtgcaggctggggggtctctgaaactctcctgtgcagcttctggaaccgaaagcattttcatcgtgatgggctggtaccgccaggctccagggaaagagcgcgagttggtcgcggcaatgtcatttggtggtggtacaaatgttacagacggcgtgaggggccgattcaccatctccagagactttgacaagaacacggtggatctgcaaatgaacaacctacagactgaggacacggccgtctattactgtaatgcagtccggtggggccctcgtgactactggggccaggggacccaggtcaccgtctcctcagcggccgcatacccgtacgacgttccggactacggttccggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 73 - protein, 1314 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGGGGGGGGQVQLQESGGGSVQAGGSLKLSCAASGTESIFIVMGWYRQAPGKERELVAAMSFGGGTNVTDGVRGRFTISRDFDKNTVDLQMNNLQTEDTAVYYCNAVRWGPRDYWGQGTQVTVSSAAAYPYDVPDYGSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 74 - DNA, 3981 bp, synthetic construct
  source 1..3981: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtggcggcgggggcgggggtggtcaggtgcagctgcaggagtctgggggaggattggtgcaggctggggcctctctgagactctcctgtgcagcctctagacgcaccggcagtagtcttaccatgggctggttccgccaggctccagggaaggagcgtgagtttgtagcagctattagccggagtggtattagaacatactacgcagactttgtgaagggccggttcaccatctccagagacaacgccaagaacacgctctatctgcaaatgaacagcctgaaacctggggacacggccgtgtattactgtgcggctaacgacaaaacatacggtagtggtcttgacgtctacacaaggcgacaagactattactactggggcccggggacccaggtcaccgtctcctcagcggccgcatacccgtacgacgttccggactacggttccggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 75 - protein, 1327 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGGGGGGGGQVQLQESGGGLVQAGASLRLSCAASRRTGSSLTMGWFRQAPGKEREFVAAISRSGIRTYYADFVKGRFTISRDNAKNTLYLQMNSLKPGDTAVYYCAANDKTYGSGLDVYTRRQDYYYWGPGTQVTVSSAAAYPYDVPDYGSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 76 - DNA, 3981 bp, synthetic construct
  source 1..3981: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtggcggcgggggcgggggtggtcaggtgcagctgcaggagtctggaggaggattggtgcaggctgggggctctctgagactctcctgtgcagcctctagacgcctcggcagtagttatgccatggcctggttccgccaggctccagggaaggagcgtgagtttgtaacagctattagccggagtggtggcagaacatactacgcagactttgtgaagggccagttcaccatctccagagacaacgccaagaacacgctgcatctgcaaatgaacagcctgaaacctgaggacacggccgtgtattactgtgcggctaacgacaaagcatacggtaccggtcttgacgtctacacaaggcgacaagactattactactggggcccggggacccaggtcaccgtctcctcagcggccgcatacccgtacgacgttccggactacggttccggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 77 - protein, 1327 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGGGGGGGGQVQLQESGGGLVQAGGSLRLSCAASRRLGSSYAMAWFRQAPGKEREFVTAISRSGGRTYYADFVKGQFTISRDNAKNTLHLQMNSLKPEDTAVYYCAANDKAYGTGLDVYTRRQDYYYWGPGTQVTVSSAAAYPYDVPDYGSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 78 - DNA, 3981 bp, synthetic construct
  source 1..3981: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtggcggcgggggcgggggtggtcaggtgcagctgcaggagtctgggggaggattggtgcaggctggggcctctctgagactctcctgtgcagcctctagacgcaccggcagtagtcttaccatgggctggttccgccaggctccagggaaggagcgtgagtttgtagcagctattagccggagtggtattagaacatactacgcagactttgtgaagggccggttcaccatctccagagacaacgccaagaacacgctctatctgcaaatgaacagcctgaaacctgaggacacggccgtgtattactgtgcggctaacgacaaaacatacggtagtggttttgacgtctacacaaggcgacaagactattactactggggcccggggacccaggtcaccgtctcctcagcggccgcatacccgtacgacgttccggactacggttccggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 79 - protein, 1327 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGGGGGGGGQVQLQESGGGLVQAGASLRLSCAASRRTGSSLTMGWFRQAPGKEREFVAAISRSGIRTYYADFVKGRFTISRDNAKNTLYLQMNSLKPEDTAVYYCAANDKTYGSGFDVYTRRQDYYYWGPGTQVTVSSAAAYPYDVPDYGSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 80 - DNA, 5649 bp, Bacillus thuringiensis
  source 1..5649: mol_type = genomic DNA
gatcttcaaatgagaaaataagggtattccgtatgggatgcctttattttggttgggaagaaggattaaaaatcaaaaatgtaaatcagatatagtccagataattttttaaagagtgtagtatattaaaaataatgttcttataacatatatgttgattttaagaaaatattttgtttaagaattcaatccatatgagtataaaaagttaaaaggcccaaaaataagttaagggaaatcaactctttaatacaaaagtttatctcaggaattctcaactatggatagcaggaagagaagtaagcacactattaacatattaggtctatttaaattaagggcatatagtgatattttataagattggttgcactttgtgcattttttcataagatgagtcatatgttttacattgtaatacagtaagaggttttagttttaaagaacaactattatgatgaaatgtggagggaacatatggagaataatattcaaaatcaatgcgtaccttacaattgtttaaataatcctgaagtagaaatattaaatgaagaaagaagtactggcagattaccgttagatatatccttatcgcttacacgtttccttttgagtgaatttgttccaggtgtgggagttgcgtttggattatttgatttaatatggggttttataactccttctgattggagcttatttcttttacagattgaacaattgattgagcaaagaatagaaacattggaaaggaaccgggcaattactacattacgagggttagcagatagctatgaaatttatattgaagcactaagagagtgggaagcaaatcctaataatgcacaattaagggaagatgtgcgtattcgatttgctaatacagacgacgctttaataacagcaataaataattttacacttacaagttttgaaatccctcttttatcggtctatgttcaagcggcgaatttacatttatcactattaagagacgctgtatcgtttgggcagggttggggactggatatagctactgttaataatcattataatagattaataaatcttattcatagatatacgaaacattgtttggacacatacaatcaaggattagaaaacttaagaggtactaatactcgacaatgggcaagattcaatcagtttaggagagatttaacacttactgtattagatatcgttgctctttttccgaactacgatgttagaacatatccaattcaaacgtcatcccaattaacaagggaaatttatacaagttcagtaattgaggattctccagtttctgctaatatacctaatggttttaatagggcggaatttggagttagaccgccccatcttatggactttatgaattctttgtttgtaactgcagagactgttagaagtcaaactgtgtggggaggacacttagttagttcacgaaatacggctggtaaccgtataaatttccctagttacggggtcttcaatcctggtggcgccatttggattgcagatgaggatccacgtcctttttatcggacattatcagatcctgtttttgtccgaggaggatttgggaatcctcattatgtactggggcttaggggagtagcatttcaacaaactggtacgaaccacacccgaacatttagaaatagtgggaccatagattctctagatgaaatcccacctcaggataatagtggggcaccttggaatgattatagtcatgtattaaatcatgttacatttgtacgatggccaggtgagatttcaggaagtgattcatggagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtacctttgatgagtgctatccaacgtatttatatcaaaaaatagatgagtcgaaattaaaaccctatactcgttatcaattaagagggtatatcgaggatagtcaagacttagaaatctatttgatccgctataatgcaaaacacgaaacagtaaatgtgctaggtacgggttctttatggccgctttcagtccaaagtccaatcagaaagtgtggagaaccgaatcgatgcgcgccacaccttgaatggaatcctgatctagattgttcctgcagagacggggaaaaatgtgcacatcattcgcatcatttctccttggacattgatgttggatgtacagacttaaatgaggacttagatgtatgggtgatattcaagattaagacgcaagatggccatgcaagactaggaaatctagagtttctcgaagagaaaccattagtcggggaagcactagctcgtgtgaaaagagcagagaaaaaatggagagataaacgtgaaaaattggaattggaaacaaatattgtttataaagaggcaaaagaatctgtagatgctttatttgtaaactctcaatatgatcaattacaagcggatacgaatattgccatgattcatgcggcagataaacgtgttcatagaattcgggaagcgtatcttccagagttatctgtgattccgggtgtaaatgtagacattttcgaagaattaaaagggcgtattttcactgcattcttcctatatgatgcgagaaatgtcattaaaaacggtgatttcaataatggcttatcatgctggaacgtgaaagggcatgtagatgtagaagaacaaaacaaccaccgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaagtctatccaaacaacacggtaacgtgtaatgattatactgcaaatcaagaagaatacgggggtgcgtacacttcccgtaatcgtggatatgacgaaacttatggaagcaattcttctgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaatagtctcatacaaaattagttttaaatatcgttttcaaatcaattgcctaagagcatcattacaaatagataagtaatttgttgtaatgaaaaacggacatcacctccattgaaacggtgagatgtccgttttactatgttattttctagtaatacatatgtacagagcaacttaattaagcagagatattttcccctatcgatgaaaatatctctgctttttctttctttattcggtatatgctttacttgtaattgaaaataaagcactaataagagtatttataggtgtttgaagttatttcagtttatttttaaaggaggtttaaaaacgttagaaagttattaaggaataatacttattagtaaattccacatatattttataattaattatgaaatatatgtataaattgaaaatgctttatttgacattacagctaagtataattttgtatgaataaaattatatctgaaaattaaataatattacagtggagggattaatatgaaactaaagaatccagataagcatcaaagtttttctagcaatgcgaaagtagataaaatctctacggattcactaaaaaatgaaacagatatagaattacaaaacattaatcatgaagattgtttgaaaatatctgagtatgaaaatgtagagccgtttgttagtgcatcaacaattcaaacaggtattagtattgcgggtaaaatacttggcaccctaggcgttccttttgcaggacaagtagctagtctttatagttttatcttaggtgagctatggcctaaggggaaaaatcaatgggaaatctttatggaacatgtagaagagattattaatcaaaaaatatcaacttatgcaagaaataaagcacttacagacttgaaaggattaggagatgccttagctgtctaccatgaatcgcttgaaagttgggttggaaatcgtaagaacacaagggctaggagtgttgtcaagagccaatatatcgcattagaattgatgttcgttcagaaactaccttcttttgcagtgtctggagaggaggtaccattattaccgatatatgcccaagctgcaaatttacatttgttgctattaagagatgcatctatttttggaaaagagtggggattatcatcttcagaaatttcaacattttataaccgtcaagtcgaacgagcaggagattattccgaccattgtgtgaaatggtatagtacaggtctaaataacttgaggggtacaaatgccgaaagctgggttcgttataatcaatttcgtaaagatatgacattaatggtacttgatttagtcgcactattcccaagctatgatacacttgtatatccaattaaaactacttctcaacttacaagagaagtatatacagacgcaattgggacagtacatccgaatgcaagttttgcaagtacgacttggtataataataatgccccttcgttctctaccatagagtctgctgttgttcgaaacccgcatctactcgattttctagaacaagttacaatttacagcttattaagtaggtggagtaacactcagtatatgaatatgtggggaggacatagacttgaattccgaacaatcggaggaatgttaaatacctcaacacaaggatc

SEQ ID NO: 81 - protein, 1174 aa, Bacillus thuringiensis; identical to SEQ ID NO: 3

SEQ ID NO: 82 - protein, 1174 aa, Bacillus thuringiensis
MENNIQNQCVPYNCLNNPEVEILNEERSTGRLPLDISLSLTRFLLSEFVPGVGVAFGLFDLIWGFITPSDWSLFLLQIEQLIEQRIETLERNRAITTLRGLADSYEIYIEALREWEANPNNAQLREDVRIRFANTDDALITAINNFTLTSFEIPLLSVYVQAANLHLSLLRDAVSFGQGWGLDIATVNNHYNRLINLIHRYTKHCLDTYNQGLENLRGTNTRQWARFNQFRRDLTLTVLDIVALFPNYDVRTYPIQTSSQLTREIYTSSVIEDSPVSANIPNGFNRAEFGVRPPHLMDFMNSLFVTAETVRSQTVWGGHLVSSRNTAGNRINFPSYGVFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEISGSDSWRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGSDTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGTFDECYPTYLYQKIDESKLKPYTRYQLRGYIEDSQDLEIYLIRYNAKHETVNVLGTGSLWPLSVQSPIRKCGEPNRCAPHLEWNPDLDCSCRDGEKCAHHSHHFSLDIDVGCTDLNEDLDVWVIFKIKTQDGHARLGNLEFLEEKPLVGEALARVKRAEKKWRDKREKLELETNIVYKEAKESVDALFVNSQYDQLQADTNIAMIHAADKRVHRIQEAYLPELSVIPGVNVDIFEELKGRIFTAFFLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTANQEEYGGAYTSRNRGYDETYGSNSSVPADYASVYEEKSYTDGRRDNPCESNRGYGDYTPLPAGYVTKELEYFPETDKVWIEIGETEGTFIVDSVELLLMEE

SEQ ID NO: 83 - protein, 1174 aa, Bacillus thuringiensis
MENNIQNQCVPYNCLNNPEVEILNEERSTGRLPLDISLSLTRFLLSEFVPGVGVAFGLFDLIWGFITPSDWSLFLLQIEQLIEQRIETLERNRAITTLRGLADSYEIYIEALREWEANPNNAQLREDVRIRFANTDDALITAINNFTLTSFEIPLLSVYVQAANLHLSLLRDAVSFGQGWGLDIATVNNHYNRLINLIHRYTKHCLDTYNQGLENLRGTNTRQWARFNQFRRDLTLTVLDIVALFPNYDVRTYPIQTSSQLTREIYTSSVIEDSPVSANIPNGFNRAEFGVRPPHLMDFMNSLFVTAETVRSQTVWGGHLVSSRNTAGNRINFPSYGVFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEISGSDSWRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGTFDECYPTYLYQKIDESKLKPYTRYQLRGYIEDSQDLEIYLIRYNAKHETVNVLGTGSLWPLSVQSPIRKCGEPNRCAPHLEWNPDLDCSCRDGEKCAHHSHHFSLDIDVGCTDLNEDLDVWVIFKIKTQDGHARLGNLEFLEEKPLVGEALARVKRAEKKWRDKREKLELETNIVYKEAKESVDALFVNSQYDQLQADTNIAMIHAADKRVHRIQEAYLPELSVIPGVNVDIFEELKGRIFTAFFLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTANQEEYGGAYTSRNRGYDETYGSNSSVPADYASVYEEKSYTDGRRDNPCESNRGYGDYTPLPAGYVTKELEYFPETDKVWIEIGETEGTFIVDSVELLLMEE

SEQ ID NO: 84 - protein, 1174 aa, Bacillus thuringiensis
MKNNIQNQCVPYNCLSNPEVEILSEERSTGRLPLDISLSLTRFLLSEFVPGVGVAFGLFDLIWGFITPSEWSLFLLQIEQLIEQRIETLERNRAITTLRGLADSYEVYLEALREWEENPNNAQLREDVRIRFANTDDALITAINNFTLTSFEIPLLSVYVQAANLHLSLLRDAVSFGQGWGLDIATVNNHYNRLINLIHRYTEHCLDTYNQGLENLRGTNTRQWSRFNQFRRELTLTVLDIVALFPNYDARAYPIQTSSQLTREIYTSSVIEDSPVSANIPNGFNRAEFGVRPPHLMDFMNSLFVTAETVRSQTVWGGHLVSSRNTAGNPINFPIYGIFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEIAGSDSWRAPMFSWTHRSADRTNIINPNIITQIPAVKAHNLHSGSTVVRGPGFTGGDLLRRTNTGTFADIRVNITGPLSQRYRVRIRYASTTDLQFFTRINGTSVNQGNFQRTMNRGGNLESGNFRTAGFSTPFSFSNAQSTFTLGTQAFSNQEVYIDRIEFVPAEVTFEAESDLERAQKAVNALFTSTSQLGLKTNVTGYHIDQVSNLVACLSDEFCLDEKRELSEKVKHAKRLSDKRNLLQDPNFRGINRQPDHGWRGSTDITIQGGDDVFKENYVTLPGTFDECYPTYLYQKIDESKLKAYTRYQLRGYIEDSQDLEIYLIRYNSKHEIVNVPGTGSLWPLSVENQIGPCGEPNRCAPHLEWNPDLHCSCRDGEKCVHHSHHFSLDIDVGCTDLNEDLGVWLIFKIKTQDGHARLGNLEFLEEEPLLGEALARVKRAEKKWRDKREKLQLETNIVYKEAKESVDALFVNSQYDRLQADTNIAMIHAADKRVHRIREAYLPELSVIPGVNAAIFEELEGRIFTAYSLYDARNVIKNGNFNNGLLCWNVKGHVDVEEQNNHRSVLVIPEWEAEVSQKVRVCPGRGYILRVTAYKEGYGEGCVTIHEIEDNTDELKFSNCVEEGYPNNTVTCNEYTMNQGVGECTDACNVRNRGYEDAYGHNPSTPVHYTTPYEEETYTDERRENPCEANKGYVNYTPLPVGYVTKELEYFPETDTVWIEIGETEGTFIVDSVELLLMEE

SEQ ID NO: 85 - protein, 1168 aa, Bacillus thuringiensis
MKNNIQNQCVPYNCLSNPEVEILSEERSTGRLPLDISLSLTRFLLSEFVPGVGVAFGLFDLIWGFITPSEWSLFLLQIEQLIEQRIETLERNRAITTLRGLADSYEVYLEALREWEENPNNAQLREDVRIRFANTDDALITAINNFTLTSFEIPLLSVYVQAANLHLSLLRDAVSFGQGWGLDIATVNNHYNRLINLIHRYTEHCLDTYNQGLENLRGTNTRQWSRFNQFRRELTLTVLDIVALFPNYDARAYPIQTSSQLTREIYTSSVIEDSPVSANIPNGFNRAEFGVRPPHLMDFMNSLFVTAETVRSQTVWGGHLVSSRNTAGNPINFPIYGIFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEIAGSDSWRAPMFSWTHRSADRTNIINPNIITQIPAVKAHNLHSGSTVVRGPGFTGGDLLRRTNTGTFADIRVNITGPLSQRYRVRIRYASTTDLQFFTRINGTSVNQGNFQRTMNRGGNLESGNFRTAGFSTPFSFSNAQSTFTLGTQAFSNQEVYIDRIEFVPAEVTFEAESDLERAQKAVNALFTSTSQLGLKTNVTGYHIDQVSNLVACLSDEFCLDEKRELSEKVKHAKRLSDKRNLLQDPNFRGINRQPDHGWRGSTDITIQGGDDVFKENYVTLPGTFDECYPTYLYQKIDESKLKAYTRYQLRGYIEDSQDLEIYLIRYNSKHEIVNVPGTGSLWPLSVENQIGPCGEPNRCAPHLEWNPDLHCSCRDGEKCVHHSHHFSLDIDVGCTDLNEDLGVWLIFKIKTQDGHARLGNLEFLEEEPLLGEALARVKRAEKKWRDKREKLQLETNIVYKEAKESVDALFVNSQYDRLQADTNIAMIHAADKRVHRIREAYLPELSVIPGVNAAIFEELEGRIFTAYSLYDARNVIKNGNFNNGLLCWNVKGHVDVEEQNNHRSVLVIPEWEAEVSQKVRVCPGRGYILRVTAYKEGYGEGCVTIHEVDNNTDELKFSNCEKEQVYPGNTVACNDYNKNHGANACSSRNRGYDESYESNSSIPADYAPVYEEEAYTDGQRGNPCEFNRGHTPLPAGYVTAELEYFPETDTVWVEIGETEGTFIVDSVELLLMEE

SEQ ID NO: 86 - protein, 1169 aa, Bacillus thuringiensis
MKNNIQNQCVPYNCLSNPEVEILSEERSTGRLPLDISLSLTRFLLSEFVPGVGVAFGLFDLIWGFITPSEWSLFLLQIEQLIEQRIETLERNRAITTLRGLADSYEVYLEALREWEENPNNAQLREDVRIRFANTDDALITAINNFTLTSFEIPLLSVYVQAANLHLSLLRDAVSFGQGWGLDIATVNNHYNRLINLIHRYTEHCLDTYNQGLENLRGTNTRQWSRFNQFRRELTLTVLDIVALFPNYDARAYPIQTSSQLTREIYTSSVIEDSPVSANIPNGFNRAEFGVRPPHLMDFMNSLFVTAETVRSQTVWGGHLVSSRNTAGNPINFPIYGVFNPGGAIWIADEDPRPFYRTLSDPVFVRGGFGNPHYVLGLRGVGFQQTGTNHTRTFRNSGTIDSLDEIPPQDNSGAPWNDYSHVLNHVTFVRWPGEIAGSDSWRAPMFSWTHRSADRTNIINPNIITQIPAVKAHNLHSGSTVVRGPGFTGGDLLRRTNTGTFADIRVNITGPLSQRYRVRIRYASTTDLQFFTRINGTSVNQGNFQRTMNRGGNLESGNFRTAGFSTPFSFSNAQSTFTLGTQAFSNQEVYIDRIEFVPAEVTFEAESDLERAQKAVNALFTSTSQLGLKTNVTGYHIDQVSNLVACLSDEFCLDEKRELSEKVKHAKRLSDKRNLLQDPNFRGINRQPDHGWRGSTDITIQGGDDVFKENYVTLPGTFDECYPTYLYQKIDESKLKAYTRYQLRGYIEDSQDLEIYLIRYNSKHEIVNVPGTGSLWPLSVENQIGPCGEPNRCAPHLEWNPDLHCSCRDGEKCVHHSHHFSLDIDVGCTDLNEDLGVWLIFKIKTQDGHARLGNLEFLEEEPLLGEALARVKRAEKKWRDKREKLQLETNIVYKEAKESVDALFVNSQYDRLQADTNIAMIHAADKRVHRIREAYLPELSVIPGVNAAIFEELEGRIFTAYSLYDARNVIKNGNFNNGLLCWNVKGHVDVEEQNNHRSVLVVPEWEAEVSQEVRVCPGRGYILRVTAYKEGYGEGCVTIHEVDNNTDELKFSSNCEKEQVYPGNTVACNDYNKNHGANACSSRNGGYDESYESNSSIPADYAPVYEEEAYTDGQRGNPCEFNRGHTPLPAGYVTAELEYFPETDTVWVEIGETEGTFIVDSVELLLMEE

SEQ ID NO: 87 - protein, 1168 aa, Bacillus thuringiensis
MKNNIQNQCVPYNCLSNPEVEILSEERSTGRLPLDISLSLTRFLLSEFVPGVGVAFGLFDLIWGFITPSEWSLFLLQIEQLIEQRIETLERNRAITTLRGLADSYEVYLEALREWEENPNNAQLREDVRIRFANTDDALITAINNFTLTSFEIPLLSVYVQAANLHLSLLRDAVSFGQGWGLDIATVNNHYNRLINLIHRYTEHCLDTYNQGLENLRGTNTRQWSRFNQFRRELTLTVLDIVALFPNYDARAYPIQTSSQLTREIYTSSVIEDSPVSANIPNGFNRAEFGVRPPHLMDFMNSLFVTAETVRSQTVWGGHLVSSRNTAGNPINFPIYGVFNPGGAIWIADEDPRPFYRTLSDPVFVRGGFGDPHYVLGLRGVGFQQTGTNHTRTFRNSGTIDSLDEIPPQDNSGAPWNDYSHVLNHVTFVRWPGEIAGSDSWRAPMFSWTHRSADRTNIINPNIITQIPAVKAHNLHSGSTVVRGPGFTGGDLLRRTNTGTFADIRVNITGPLSQRYRVRIRYASTTDLQFFTRINGTSVNQGNFQRTMNRGDNLESGNFRTAGFSTPFSFSNAQSTFTLGTQAFSNQEVYIDRIEFVPAEVTFEAESDLERAQKAVNALFTSTSQLGLKTNVTGYHIDQVSNLVACLSDEFCLDEKRELSEKVKHAKRLSDKRNLLQDPNFRGINRQPDHGWRGSTDITIQGGDDVFKENYVTLPGTFDECYPTYLYQKIDESKLKAYTRYQLRGYIEDSQDLEIYLIRYNSKHEIVNVPGTGSLWPLSVENQIGPCGEPNRCAPHLEWNPDLHCSCRDGEKCVHHSHHFSLDIDVGCTDLNEDLGVWLIFKIKTQDGHARLGNLEFLEEEPLLGEALGRVKRAEKKWRDKREKLQLETNIVYKEAKESVDALFVNSQYDRLQADTNIAMIHAADKRVHRIREAYLPELSVIPGVNAAIFEELEGRIFTAYSLYDARNVIKNGNFNNGLLCWNVKGHVDVEEQNNHRSVLVVPEWEAEVSQEVRVCPGRGYILRVTAYKEGYGEGCVTIHEVDNNTDELKFSNCEKEQVYPGNTVACNDYNKNHGANACSSRNRGYDESYESNSSIPADYAPVYEEEAYTDGQRGNPCEFNRGHTPLPAGYVTAELEYFPETDTVWVEIGETEGTFIVDSVELLLMEE

SEQ ID NO: 88 - protein, 1174 aa, Bacillus thuringiensis; identical to SEQ ID NO: 84

SEQ ID NO: 89 - protein, 1168 aa, Bacillus thuringiensis
MENNIQNQCVPYNCLSNPEVEILSEERSTGRLPLDISLSLTRFLLSEFVPGVGVAFGLFDLIWGFITPSEWSLFLLQIEQLIEQRIETLERNRAITTLRGLADSCEVYLEALREWEENPNNAQLREDVRIRFANTDDALITAINNFTLTSFEIPLLSVYVQAANLHLSLLRDAVSFGQGWGLDIATVNNHYNRLINLIHRYTEHCLDTYNQGLENLRGTNTRQWSRFNQFRRELTLTVLDIVALFPNYDARAYPIQTSSQLTREIYTSSVIEDSPVSANIPNGFNRAEFGVRPPHLMDFMNSLFVTAETVRSQTVWGGHLVSSRNTAGNPINFPIYGVFNPGGAIWIADEDPRPFYRTLSDPVFVRGGFGNPHYVLGLRGVGFQQTGTNHTRTFRNSGTIDSLDEIPPQDNSGAPWNDYSHVLNHVTFVRWPGEIAGSDSWRAPMFSWTHRSADRTNIINPNIITQIPAVKAHNLHSGSTVVRGPGFTGGDLLRRTNTGTFADIRVNITGPLSQRYRVRIRYASTTDLQFFTRINGTSVNQGNFQRTMNRGDNLESGNFRTAGFSTPFSFSNAQSTFTLGTQAFSNQEVYIDRIEFVPAEVTFEAESDLERAQKAVNALFTSTSQLGLKTNVTGYHIDQVSNLVACLSDEFCLDEKRELSEKVKHAKRLSDKRNLLQDPNFRGINRQPDHGWRGSTDITIQGGDDVFKENYVTLPGTFDECYPTYLYQKIDESKLKAYTRYQLRGYIEDSQDLEIYLIRYNSKHEIVNVPGTGSLWPLSVENQIGPCGEPNRCAPHLEWNPDLHCSCRDGEKCVHHSHHFSLDIDVGCTDLNEDLGVWLIFKIKTQDGHARLGNLEFLEEEPLLGEALARVKRAEKKWRDKREKLQLETNIVYKEAKESVDALFVNSQYDRLQADTNIAMIHAADKRVHRIREAYLPELSVIPGVNAAIFEELEGRIFTAYSLYDARNVIKNGNFNNGLLCWNVKGHVDVEEQNNHRSVLVVPEWEAEVSQEVRVCPGRGYILRVTAYKEGYGEGCVTIHEVDNNTDELKFSSCEKEQVNPGNTVACNDYNKNHGANACSSRSRGYDESYESNSSIPADYAPVYEEEAYTDGQRGNPCEFNRGHTPLPAGYVTAELEYFPETDTVWVEIGETEGTFIVDSVELLLMEE

SEQ ID NO: 90 - protein, 1174 aa, Bacillus thuringiensis
MENNIQNQCVPYNCLSNPEVEILSEERSTGRLPLDISLSLTRFLLSEFVPGVGVAFGLFDLIWGFITPSEWSLFLLQIEQLIEQRIETLERNRAITTLRGLADSYEVYLEALREWEENPNNAQLREDVRIRFANTDDALITAINNFTLTSFEIPLLSVYVQAANLHLSLLRDAVSFGQGWGLDIATVNNHYNRLINLIHRYTEHCLDTYNQGLENLRGTNTRQWSRFNQFRRELTLTVLDIVALFPNYDAGAYPIQTSSQLTREIYTSSVIEDSPVSANIPNGFNRAEFGVRPPHLMDFMNSLFVTAETVRSQTVWGGHLVSSRNTAGNPINFPIYGIFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEIAGSDSWRAPMFSWTHRSADRTNIINPNIITQIPAVKAHNLHSGSTVVRGPGFTGGDLLRRTNTGTFADIRVNITGPLSQRYRVRIRYASTTDLQFFTRINGTSVNQGNFQRTMNRGGNLESGNFRTAGFSTPFSFSNAQSTFTLGTQAFSNQEVYIDRIEFVPAEVTFEAESDLERAQKAVNALFTSTSQLGLKTNVTGYHIDQVSNLVACLSDEFCLDEKRELSEKVKHAKRLSDKRNLLQDPNFRGISRQPDHGWRGSTDITIQGGDDVFKENYVTLPGTFDECYPTYLYQKIDESKLKAYTRYQLRGYIEDSQDLEIYLIRYNSKHEIVNVPGTGSLWPLSVENQIGPCGEPNRCAPHLEWNPDLHCSCRDGEKCVHHSHHFSLDIDVGCTDLNEDLGVWLIFKIKTQDGHARLGNLEFLEEEPLLGEALARVKRAEKKWRDKREKLQLETNIVYKEAKESVDALFVNSQYDRLQADTNIAMIHAADKRVHRIREAYLPELSVIPGVNAAIFEELEGRIFTAYSLYDARNVIKNGNFNNGLLCWNVKGHVDVEEQNNHRSVLVIPEWEAEVSQKVRVCPGRGYILRVTAYKEGYGEGSVTIHEIEDNTDELKFSNCVEEGYPNNTVTCNEYTMNQGVGECTDACNVRNRGYEDAYGHNPSTPVHYTTPYEEETYTDERRENPCEANKGYVNYTPLPVGYVTKELEYFPETDKVWVEIGETEGTFIVDSVELLLMEE

SEQ ID NO: 91 - protein, 1177 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVELLLMEE

SEQ ID NO: 92 - protein, 13 aa, synthetic construct
AAAYPYDVPDYGS

SEQ ID NO: 93 - DNA, 3963 bp, synthetic construct
  source 1..3963: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaaggcggtgaatgcgctgtttacttctataaaccaaatagggataaaaacagatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcaaaggcatcaataggcaactagaccgtggttggagaggaagtacggatattaccatccaaagaggagatgacgtattcaaagaaaattatgtcacactaccaggtggcggcgggggcgggggtggtcaggtgcagctgcaggagtctgggggaggcttggtgcaggctgggggatctctgacactctcctgtacagcctctggaagcatcactagtatcaatgccatgggcttctaccgccagtacgcagagaaccagcgtgagttggtcgcatcgattactaaagatggcgccccaagctatattcgctccgtgaagggccgattcaccatctccagagacaacttcaagaacacgctgtatctgcagatgaacagcctgaaacctgaggacacggccgtttattactgcgcagcgtcaaggatgtacagcgacactccttcgaagcaatcgattgccttctggggccaggggacccaggtcaccgtctcctcagcggccgcatacccgtacgacgttccggactacggttccggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattactccttatggaggaa

SEQ ID NO: 94 - protein, 1321 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGGGGGGGGQVQLQESGGGLVQAGGSLTLSCTASGSITSINAMGFYRQYAENQRELVASITKDGAPSYIRSVKGRFTISRDNFKNTLYLQMNSLKPEDTAVYYCAASRMYSDTPSKQSIAFWGQGTQVTVSSAAAYPYDVPDYGSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRDNPCESNRGYGDYTPLPAGYVTKELEYFPETDKVWIEIGETEGTFIVDSVELLLMEE

SEQ ID NO: 95 - DNA, 369 bp, synthetic construct
  source 1..369: mol_type = other DNA
caggtgcagctgcaggagtctgggggaggcttggtgcaggctgggggatctctgacactctcctgtacagcctctggaagcatcactagtatcaatgccatgggcttctaccgccagtacgcagagaaccagcgtgagttggtcgcatcgattactaaagatggcgccccaagctatattcgctccgtgaagggccgattcaccatctccagagacaacttcaagaacacgctgtatctgcagatgaacagcctgaaacctgaggacacggccgtttattactgcgcagcgtcaaggatgtacagcgacactccttcgaagcaatcgattgccttctggggccaggggacccaggtcaccgtctcctca

SEQ ID NO: 96 - protein, 123 aa, synthetic construct
QVQLQESGGGLVQAGGSLTLSCTASGSITSINAMGFYRQYAENQRELVASITKDGAPSYIRSVKGRFTISRDNFKNTLYLQMNSLKPEDTAVYYCAASRMYSDTPSKQSIAFWGQGTQVTVSS